(19) United States
(12) Patent Application Publication
(10) Pub. No.: US 2007/0123592 A1
(43) Pub. Date: May 31, 2007

(54) Terephthalamide peptidomimetic compounds and methods

(75) Inventors: Andrew D. Hamilton, Guilford, CT (US); Hang Yin, Philadelphia, PA (US)

Correspondence Address: COLEMAN SUDOL SAPONE, P.C., 714 COLORADO AVENUE, BRIDGE PORT, CT, 06605-1601, US

(73) Assignee: YALE UNIVERSITY, New Haven, CT (US)

(21) Appl. No.: 10/588,478

(22) PCT Filed: Feb. 22, 2005
(86) PCT No.: PCT/US05/05557

§ 371 Date: Oct. 2, 2006

Related U.S. Application Data
(60) Provisional application No. 60/546,111, filed on Feb. 19, 2004.

(51) Int. Cl. A61K 31/195

(52) U.S. Cl. 514/563; 562/450; 562/426; 562/439

(57) ABSTRACT

The present invention relates to compounds and pharmaceutical compositions based upon terephthalamide which are proteomimetic and to methods for inhibiting the interaction of an alpha-helical protein with another protein or binding site. Methods for treating diseases or conditions which are modulated through interactions between alpha helical proteins and their binding sites are other aspects of the invention. Methods of inhibiting the binding of proteins to their binding sites are other aspects of the present invention.

### RELATED APPLICATIONS

This application claims the priority benefit of provisional application 60/289,640, entitled “Terephthalamide Derivatives as Mimetics of the Helical Region Structure and functional mimics of a helix”, filed Feb. 19, 2004.

### FIELD OF THE INVENTION

The present invention relates to compounds and pharmaceutical compositions based upon terephthalamide which are proteomimetic and to methods for inhibiting the interaction of an alpha-helical protein with another protein or binding site. Methods for treating diseases or conditions which are modulated through interactions between alpha helical proteins and their binding sites are other aspects of the invention. Methods of inhibiting the binding of proteins to their binding sites are other aspects of the present invention.

### BACKGROUND OF THE INVENTION

Proteins in the B-cell lymphoma-2 (Bcl-2) family play a critical role in determining whether a cell survives or dies through a programmed cell death known as apoptosis.¹ The Bcl-2 protein family, comprised of both pro-apoptotic and anti-apoptotic members, acts as a checkpoint downstream of the tumor suppressor protein p53,² and upstream of mitochondrial rupture and caspase cysteine proteases, which transduce the apoptotic signal.¹ Previous studies showed that oncogenic mutations induced apoptosis defects through a Bcl-2 dependent pathway.³ Overexpression of the anti-apoptotic proteins, such as Bcl-2 and Bcl-x_{L}, can inhibit the potency of many currently available anticancer drugs by blocking the apoptotic pathway.⁴ All of the pro-apoptotic subfamily proteins possess the minimal death domain BH3. These molecules (Bak, Bax, Bad, Bid) are able to induce apoptosis through heterodimerization with the anti-apoptotic Bcl-2 family members.⁵ Several low-molecular-weight inhibitors of Bcl-2 (Bcl-x_{L}) have been identified by screening diverse chemical libraries.^{6, 7} The rational design of agents that directly mimic the death-promoting region, the BH3 domain of the pro-apoptotic subfamily of Bcl-2 proteins, is an important alternative to screening as it allows structure-based optimization of initial hits.⁸

The development of small molecule modulators of protein-protein interactions is regarded as a challenging goal since the large interfaces involved, typically around 1600 Å² of buried area (around 170 atoms), pose a serious hurdle for any small molecule to be competitive.⁹ The binding regions of protein partners are often discontiguous and thus cannot be mimicked by simple synthetic peptides with linear or extended conformations. Conventional methods for identifying inhibitors of protein-protein interactions require much input in the preparation and screening of a chemical library in order to discover lead compounds. An alternative approach is to design synthetic recognition scaffolds that reproduce features of the protein secondary structure. We have previously reported functionalized terphenyls as mimetics of α-helices.^{10, 11} However, the challenging syntheses and physical properties of terphenyls prompted us to search for simpler scaffolds that could similarly mimic the side chain presentation on an α-helix. ¹² We have recently reported a group of Bcl-x_{L} inhibitors based on a terephthalamide scaffold, designed to mimic the α-helical region of the Bak peptide.¹³ Using a fluorescence polarization assay, we have observed high in vitro inhibition potencies in disrupting the Bcl-x_{L}/Bak BH3 domain complex and a significant improvement in water solubility relative to the terphenyl derivatives.

In the present application, we disclose an expanded structure-affinity study that demonstrates a correlation between the potency of the Bcl-x_{L}/Bak disruption and the size of side chains on these molecules. Computational docking simulations and NMR experiments are used to confirm the binding mode of the terephthalamide inhibitors and suggest that the binding cleft for the Bak BH3 domain on the surface of Bcl-x_{L} is the target area for the synthetic inhibitors. Treatment of human HEK293 cells with a terephthalamide derivative resulted in inhibition of the association of Bcl-x_{L} with Bax in whole cells.

### OBJECTS OF THE INVENTION

It is an object of the invention of the present invention to provide novel compounds which exhibit proteomimetic characteristics.

It is another object of the invention to provide pharmaceutical compositions based upon the compounds according to the present invention which are useful to treat disease states or conditions which are modulated through the interaction of an α-helix protein with another protein or binding site for the protein and in particular a Bak peptide.

It is still another object of the invention to provide methods for treating disease states or conditions which are modulated through the interaction of an α-helical protein with another protein or binding site for the protein, especially those disease states which are mediated through interaction of the Bak peptide with BcI-xL or which mimic the death-promoting region BH3 of BcI-2 proteins.

It is yet another object of the invention to provide compounds, methods and compositions for the treatment of cancer alone, or in combination with at least one other anti-cancer agent.

These and/or other objects of the present invention may be readily gleaned from the description of the invention which follows.

### DESCRIPTION OF THE FIGURES

FIG. 1 shows a chemical synthetic scheme for producing certain terephthalamide derivative compounds according to the present invention. The following represents the individual steps which are presented in the synthetic scheme: (a) 2-Iodopropane, K₂CO₃, acetone, reflux; (b) NaNO₂, H₂SO₄, MeOH, H₂O, 0° C.; (c) KI, Cu (bronze), reflux; (d) Tributyl(vinyl)tin, Pd(PPh₃)₄, toluene, reflux; (e) NaOH (aq.), MeOH; (f) (COCl)₂, DMF, CH₂Cl₂; (g) (iPr)₂NH, CH₂Cl₂; (h) OsO₄, NaIO₄, tBuOH: CCl₄: H₂O (2:1:1); (i) Pyridinium dichromate, DMF; (j) L-Leucine methyl ester hydrochloride, 1-hydroxybenzotriazole hydrate, 1-[3-(dimethylamino)propyl]-3-ethylcarbodiimide hydrochloride; (k) KOH, MeOH.

FIG. 2 shows a chemical synthetic scheme for producing certain terephthalamide derivative compounds according to the present invention. The following represents the individual steps which are presented in the synthetic scheme: (a) (COCl)₂, DMF, CH₂Cl₂; (b) (iPr)₂NH, CH₂Cl₂; (c) SnCl₂, EtOAc, 0° C.; (d) Acetone, Zn, CH₃CO₂H; (e) NaOH (aq.), MeOH; (f) L-Leucine methyl ester hydrochloride, 1-hydroxybenzotriazole hydrate, 1-[3-(dimethylamino)propyl]-3-ethylcarbodiimide hydrochloride; (g) KOH, MeOH.

FIG. 3 shows a chemical synthetic sheme for producing certain terephthalamide derivative compounds according to the present invention. The following represents the individual steps which are presented in the synthetic scheme: (a) NaOH (aq.), MeOH; (b) (COCl)₂, DMF, CH₂Cl₂; (c) 2-Isobutylamino-propionic acid methyl ester (3-19), CH₂Cl₂; (d) OSO₄, NaIO₄, tBuOH: CCl₄: H₂O (2:1:1); (e) Pyridinium dichromate, DMF; (f) L-Leucine methyl ester hydrochloride, 1-hydroxybenzotriazole hydrate, 1-[3-(dimethylamino)propyl]-3-ethylcarbodiimide hydrochloride; (g) KOH, MeOH.

FIG. 4 A shows the energy minimized Z- and E- isomers of compound 3-22.

FIG. 4B shows the ROESY ¹H-¹H NMR experiments which evidenced cross peaks corresponding to the chemical exchange of H_{c}.

### SUMMARY OF THE INVENTION

The present invention relates to a compound according to the chemical structure I: 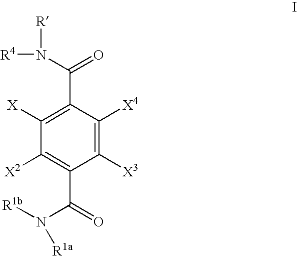
where X is H, halogen (F, Cl, Br, I), R, OR, SR or NR^{c}R^{d};
X², X³ and X⁴ are each independently selected from H, halogen, OH, R^{e} or OR^{e},
R⁴ is H, an unsubstituted or substituted C₁-C₈ alkyl or alkene (preferably a C₁-C₃ alkyl or alkanol), an unsubstituted or substituted C₁-C₆ alkylene amine (wherein the amine group where substituted is substituted with one or two C₁-C₄ alkyl groups);
R′ is H, an unsubstituted or substituted C₁-C₈ alkyl or alkene (preferably a C₁-C₃ alkyl or alkanol), an unsubstituted or substituted C₁-C₆ alkylene amine (wherein the amine group where substituted is substituted with one or two C₁-C₄ alkyl groups), or a 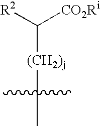
group,
where Rⁱ is H or C₁-C₆ (preferably C₁-C₃) alkyl; j is 0, 1 or 2 (preferably 0);
R² is independently H, an unsubstituted or substituted hydrocarbon, preferably a C₁-C₆ alkyl or alkene group, unsubstituted or substituted aryl, including benzyl and naphthyl, unsubstituted or substituted alkylenearyl or alkylaryl, (preferably alkylene phenyl and alkylphenyl containing from 1 to 3 substitutents on the phenyl moiety), unsubstituted or substituted alkoxy (preferably C₁-C₆), unsubstitued or substituted ester (including an alkyl or aryl ester or an alkylene ester wherein said ester group preferably comprises a C₁-C₆ alkyl or aryl, preferably benzyl or phenyl group), an unsubstituted or substituted alkanol (preferably C₁-C₆), an unsubstituted or substituted alkanoic acid (preferably C₁-C₆), an unsubstituted or substituted thioester (preferably a C₁-C₆ alkyl/C₁-C₆ alkylene thioester), an unsubstituted or substituted thioether (preferably a C₁-C₆ alkyl/C₁-C₆ alkylene thioether, more preferably an allylalkylene thioether, preferably a methyl ethylene thioether such as a methionine thioether), an unsubstituted or substituted amine (including an alkylamine and dialkylamine, preferably C₁-C₆ alkyl), an unsubstituted or substituted alkylamide (preferably, C₁-C₆ alkyl), an substituted or unsubstituted alkylene amide (preferably C₁-C₆ alkylene which may be substituted on the amine groups of the amide, preferably with alkyl groups), an unsubstituted or unsubstituted alkyleneamine (preferably C₁-C₆ alkylene), alkyleneguanidine (preferably C₁-C₆ alkylene);
or R′ together with the nitrogen atom to which R′ is attached form an amino acid residue, preferably an α-amino acid residue when j is 0 and the amino acid residue is even more preferably obtained or derived from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine or valine;
R is H, an unsubstituted or substituted C₁-C₁₀ alkyl or acyl group (preferably a C₁-C₄ alkyl or acyl group), an unsubstituted or substituted aryl, heteroaryl, allylene aryl (preferably, C₁-C₆ alkylene aryl) or alkylene heteroaryl (preferably, C₁-C₆ alkyleneheteroaryl) group;
R^{c} and R^{d} are each independently H, C₁-C₆ alkyl (preferably C₁-C₃ alkyl) or a C₁-C₆ alkanol or a C₁-C₆ acyl group with the proviso that if one of R^{c} or R^{d} is an acyl group, the other of R^{c} or R^{d} cannot also be an acyl group;
R^{e} is an unsubstituted or substituted C₁-C₆ alkyl or acyl group, or an unsubstituted or substituted aryl or alkylene aryl group;
R^{1a} and R^{1b} are each independently H, unsubstituted or substituted C₁-C₈ alkyl or alkene (preferably a C₁-C₃ alkyl or alkanol), an unsubstituted or substituted aryl or alkylene aryl group (preferably benzyl or phenyl), an unsubstited or substituted C₁-C₆ alkylene amine (wherein the amine group where substituted is substituted with one or two C₁-C₄ alkyl groups), or a 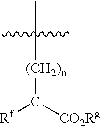
group;
Where R^{g} is H or C₁-C₆ (preferably C₁-C₃) alkyl;
n is 0, 1 or 2 (preferably 0); and
R^{f} is H, an unsubstituted or substituted hydrocarbon, preferably an alkyl or alkene group, unsubstituted or substituted aryl, including benzyl and naphthyl, unsubstituted or substituted alkylenearyl or alkylaryl, (preferably alkylene phenyl and alkylphenyl containing from 1 to 3 substituteents on the phenyl moiety), unsubstituted or substituted alkoxy, unsubstitued or substituted ester (including an alkyl or aryl ester or an alkylene ester wherein said ester group preferably comprises a C₁-C₆ alkyl or aryl, preferably benzyl or phenyl group), an unsubstituted or substituted alkanol, an unsubstituted or substituted alkanoic acid, an unsubstituted or substituted thioester (preferably a C₁-C₆ alkyl/C₁-C₆ alkylene thioester), an unsubstituted or substituted thioether (preferably a C₁-C₆ alkyl/C₁-C₆ alkylene thioether, more preferably an alkylalkylene thioether, preferably a methyl ethylene thioether such as methionine thioether), an unsubstituted or substituted amine (including an alkylamine and dialkylamine, preferably C₁-C₆ alkyl), an unsubstituted or substituted alkylamide (preferably, C₁-C₆ alkyl), an substituted or unsubstituted alkylene amide (preferably C₁-C₆ alkylene which may be substituted on the amine groups of the amide, preferably with alkyl groups), an unsubstituted or unsubstituted alkyleneamine (preferably C₁-C₆ alkylene), alkyleneguanidine (preferably C₁-C₆ alkylene); or
R^{1a} and R^{1b}, together with the nitrogen atom to which R^{1a} and R^{1b} are attached, form an amino acid residue, preferably an α-amino acid residue when n is 0 wherein the amino acid residue is preferably derived or obtained from alanine, arginine, asparagine, aspartic acid, cysteine, glutamine, glutamic acid, glycine, histidine, isoleucine, leucine, lysine, methionine, phenylalanine, praline, serine, threonine, tryptophan, tyrosine or valine; and pharmaceutically acceptable salts, thereof.

In preferred aspects of the present invention, R⁴ is H, R′ is preferably H, a C₁-C₄ alkyl group, an unsubstituted or substituted phenyl group or more preferably a 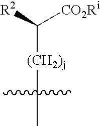
group,
where j is 0, R¹ is H, and
R² is an unsubstituted or substituted alkyl or aryl group, an unsubstituted or substituted alkoxy or ester group, an unsubstituted or substituted alkanol or alkanoic acid, an unsubstituted or substituted C₁-C₆ thioether, an unsubstituted or substituted amine, an unsubstituted or substituted alkylamide or alkylene amide or an alkyleneguanidine group; or
R′ together with the nitrogen atom to which it is attached forms an α-amino acid residue wherein the amino acid residue is preferably derived from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine or valine, and pharmaceutically acceptable salts thereof;
X is preferably a hydrogen bond acceptor group, and is preferably an OR group, more preferably an O-alkyl group or O-aryl group;
R^{1a} and R^{1b} are each independently H, unsubstituted or substituted C₁-C₄ alkyl or together with the nitrogen atom to which R^{1a} and R^{1b} are attached form an α-amino acid residue wherein the amino acid residue is preferably derived or obtained from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine or valine, and pharmaceutically acceptable salts thereof.

Compounds according to the present invention may be used as active agents in pharmaceutical compositions as agonists or inhibitors of α-helical proteins in their interactions with proteins (such as receptors, enzymes, other proteins) or other binding sites, said compositions comprising an effective amount of one or more of the compounds disclosed above, formulated as a pharmaceutical dosage form, optionally in combination with a pharmaceutically acceptable carrier, additive or excipient. Pharmaceutical compositions according to the present invention may be used in the treatment of cancer (as, for example, a suppressor of Mdm2/p53 tumor, to inhibit BcL protein family/Bak protein family or AP-1 transcription factor/DNA complex), proliferative diseases including, for example, psoriasis, genital warts and hyperproliferative keratinocyte diseases including hyperkeratosis, ichthyosis, keratoderma or lichen planus, neuropeptide Y receptor interactions, including the resulting hypertension and and neuronal/neurological effects (to facilitate neuromodulation through, for example, inhibition of calmodulin binding on calmodulin dependent phosphodiesterase including PDE1A, PDE1B and PDE1C, among others), neurodegenerative diseases including Alzheimer's disease and Parkinson's disease, Herpes simplex virus infections (HSV, through inhibition of the HSV VP 16/human TAF1131 HSV infection complex), HIV infections (through inhibition of HIVp7 nuclear capsid protein/RNA interaction or alternatively, through inhibition of the REV protein RNA complex), asthma, hypertension, cancer and autoimmune diseases (through immunomodulation, for example, by inhibition or modulation of interleukin/receptor interaction), numerous viral infections other than HIV or HSV through inhibition of ribonucleotide reductase dimerization, or to modulate nuclear receptor/coactivator protein complex interaction (eg. estrogen receptor for anticancer therapy) and to disrupt G protein coupled receptor (GPCR) function (through displacement of one of the helixes and disruption of the helix packing interactions or alternatively, by blocking the interacton of the ligand with GPCR, e.g. where the ligand contains a key helix binding domain (e.g. GCSF, calcitonin, interleukins, parathyroid hormones, among others).

In other aspects of the present invention, certain compounds according to the present invention may be used as agonists or antagonists in binding assays, as analytical agents, as agents to be used to isolate or purify proteins, and as intermediates in the synthesis of further peptidomimetic agents, among other uses.

The present invention also relates to methods of treating patients in need thereof for conditions or disease states which are modulated through interactions between alpha helical proteins and other proteins or binding sites are other aspects of the invention. Thus, in the method aspect of the present invention, pharmaceutical compositions comprising α-helical protein agonists or antagonists may be used to treat any condition or disease state in which α-helical proteins modulate their activity through a receptor or other binding site. In particular, the method aspect of the present invention relates to the inhibition of protein binding to binding sites within the patient in order to effect a biological/pharmacological result. Compounds according to the present invention may be used as proteomimetics to inhibit the interaction between a native α helical protein (i.e., a natural α helical protein normally found in a patient) and its binding site. Preferred compounds according to the present invention may be used to disrupt or compete with the binding of a number of proteins including, for example, calmodulin (CaM) with binding sites on smooth muscle light chain kinase (smMLCK) or phosphodiesterase (PDE1A, PDE1B, PDE1C) with resulting neuromuscular and neuronal (among other) effects in the treating of disease states or conditions, gp41 (HIV) and other viruses such as HSV or HBV, for the viral invasive binding cites in CD4 and/or other hematopoietic cells, genital/mucosal cells, among others (HSV)and hepatocytes (HBV), among numerous others and pro-apoptotic Bak- and/or Bad-proteins, for their binding interaction with Bcl-x_{L} protein in a preferred treatment for cancer.

Thus, the present application is directed to the treatment of disease states or conditions which are modulated through interactions between α-helical proteins and other proteins or binding sites of the α-helical proteins preferably selected from the group consisting of viral infections (including Hepatitis B virus (HBV) infections, human immunodeficiency virus (HIV) infections or conditions associated with such infections (AIDS), Herpes Simplex virus infections (HSV) infections, tumors and/or cancer, proliferative diseases including psoriasis, genital warts and hyperproliferative keratinocyte diseases including hyperkeratosis, ichthyosis, keratoderma, lichen planus, hypertension, neuronal disorders by promoting neuromodulation including, for example, attention deficit disorder, memory loss, language and learning disorders, asthma, autoimmune diseases including lupus (lupus erythematosus), multiple sclerosis, arthritis, including rheumatoid arthritis, rheumatic diseases, fibromyalgia, Sjögren's disease and Grave's disease and neurodegenerative diseases including Alzheimer's disease and Parkinson's disease, said method comprising administering to a patient in need thereof an effective amount of a pharmaceutical composition comprising any one or more of the compounds previously described above.

Definitions: The following definitions shall be used to describe the present invention.

“Patient” refers to a mammal, preferably a human, in need of treatment or therapy to which compounds according to the present invention are administered in order to treat a condition or disease state modulated through the binding of an α-helical protein with a binding site.

“Modulated” means, with respect to disease states or conditions modulated through binding of α-helical proteins to binding sites, that the binding or lack or absence of binding of an α-helical protein to a binding site produces or will produce, either directly or indirectly, a condition or disease state which is sub-optimal and in many cases, debilitating and even life threatening.

The term “compound” is used herein to refer to any specific chemical compound disclosed herein. Within its use in context, the term generally refers to a single compound, but in certain instances may also refer to stereoisomers and other positional isomers and/or optical isomers (including racemic mixtures) of disclosed compounds. The compounds of this invention include all stereoisomers where relevant (e.g., cis and trans isomers) and all optical isomers of the present compounds (eg., R and S enantiomers), as well as racemic, diastereomeric and other mixtures of such isomers, as well as all polymorphs of the present compounds, where applicable.

“Sterically and electronically similar” refers to synthetic substituents on chemical cages or scaffolds according to the present invention which mimic the steric and/or electronic physicochemical characteristics of substituents on α carbons in natural α helical proteins. While not necessarily identical to the natural substituents, substituents which are sterically and electronically similar to the natural substituents promote the binding of synthetic compounds according to the present invention to α helical protein binding sites.

“Chemical cages or scaffords” according to the present invention represent terephthalamide derivatives as otherwise disclosed herein in which a terephthalamide chemical moiety is central to substituents on the 1 and 4 amide positions and are substituted with groups bound to the two amide groups, as well as other positions of the phenyl group. The present compounds form pepidomimetics which are useful for mimicking the chemical and pharmacological effects of proteins and exhibit utility for treating a number of conditions and disease states. These chemical cages are generally substituted with any number of substituents, preferably those which mimic natural substituents on a carbons (from the amino acids) of α helical proteins.

The term “hydrogen bond acceptor group” refers to a group, such as a O-alkyl group or other group which has sufficient electron density to form a hydrogen bond with a hydrogen atom on an adjacent chemical moiety.

“Hydrocarbon” refers to any monovalent radical containing carbon and hydrogen, which may be straight or branch-chained or cyclic in nature. Hydrocarbons include linear, branched and cyclic hydrocarbons, including alkyl groups, alkylene groups, unsaturated hydrocarbon groups, both substituted and unsubstituted.

“Alkyl” refers to a fully saturated monovalent radical containing carbon and hydrogen, and which may be cyclic, branched or a straight chain. Examples of alkyl groups are methyl, ethyl, n-butyl, n-hexyl, n-heptyl, n-octyl, +isopropyl, 2-methylpropyl, cyclopropyl, cyclopropylmethyl, cyclobutyl, cyclopentyl, cyclopentylethyl, cyclohexylethyl and cyclohexyl. Preferred alkyl groups are C₁-C₆ alkyl groups. “Alkylene” refers to a fully saturated hydrocarbon which is divalent (may be linear, branched or cyclic) and which is optionally substituted. Other terms used to indicate substitutuent groups in compounds according to the present invention are as conventionally used in the art. Thus, the term alkylene aryl includes alkylene phenyl such as a benzyl group or ethylene phenyl group, alkylaryl, includes alkylphenyl such a phenyl group which has alkyl groups as substituents, etc.

“Aryl” refers to a substituted or unsubstituted monovalent aromatic radical having a single ring (e.g., benzene) or multiple condensed rings (e.g., naphthyl, anthracenyl, phenanthryl) and can be can be bound to compound according to the present invention at any position on the ring(s). Other examples of aryl groups include heterocyclic aromatic ring systems “heteroaryl” groups having one or more nitrogen, oxygen, or sulfur atoms in the ring, such as imidazole, furyl, pyrrole, pyridyl, indole and fused ring systems, among others, which may be substituted or unsubstituted.

“Alkoxy” as used herein refers to an alkyl group bound through an ether linkage; that is, an “alkoxy” group may be represented as —O-alkyl where alkyl is as defined above. A “lower alkoxy” group refers to an alkoxy group containing one to six, more preferably one to four, carbon atoms.

The term “cyclic” shall refer to a carbocyclic or heterocyclic group, preferably a 5- or 6-membered ring. A heterocyclic ring shall contain up to four atoms other than carbon selected from nitrogen, sulfur and oxygen.

The term “effective amount” refers to the amount of a selected compound which is effective within the context of its use or administration. In the case of therapeutic methods according to the present invention, the precise amount required will vary depending upon the particular compound selected, the age and weight of the subject, route of ad-ministration, and so forth, but may be easily determined by routine experimentation.

The term “substituted” shall mean substituted at a carbon (or nitrogen) position with, in context, hydroxyl, carboxyl, halogen, thiol, an alkyl group (preferably, C₁-C₆), alkoxy group (preferably, C₁-C₆ alkyl or aryl), ester (preferably, C₁-C₆ alkyl or aryl) including alkylene ester (such that attachment is on the alkylene group, rather than at the ester function which is preferably substituted with a C₁-C₆ alkyl or aryl group), thioether (preferably, C₁-C₆ alkyl or aryl), thioester (preferably, C₁-C₆ alkyl or aryl), (preferably, C₁-C₆ alkyl or aryl), halogen (F, Cl, Br, I), nitro or amine (including a five- or six-membered cyclic alkylene amine, preferably, a C₁-C₆ alkyl amine or C₁-C₆ dialkyl amine), alkanol (preferably, C₁-C₆ alkyl or aryl), or alkanoic acid (preferably, C₁-C₆ alkyl or aryl). Preferably, the term “substituted” shall mean within its context of use alkyl, alkoxy, halogen, hydroxyl, carboxylic acid, nitro and amine (including mono- or di- alkyl substituted amines). The term unsubstituted shall mean substituted with one or more H atoms.

The term “amino acid residue” means an amino acid radical which is obtained or derived from an amino acid as otherwise described herein. In many instances, but not exclusively, the amino acid residues are formed from the amine group from an alpha, beta or gamma amino acid reacting with an activated acid or other group and forming an amide group of the phthalamide compounds according to the present invention.

The term “binding site” refers to a site at which an -helical protein binds and—elicits some response or action at that binding site, which action may be direct or indirect. Compounds according to the present invention will also bind at the binding site of the—helical binding site in an agonistic or antagonistic manner. The binding site may be another protein, a receptor (such as a cell surface receptor or a G-protein coupled receptor), signaling proteins, proteins involved in apoptotic pathways (especially neuronal apoptosis), active sites and regulatory domains of enzymes, growth factors, DNA, RNA (including polynucleotides and oligonucleotides), viral fusion proteins and viral coat proteins, among numerous others.

The term “pharmaceutically acceptable carrier” refers to carrier, additive or excipient which is not unacceptably toxic to the subject to which it is administered. Pharmaceutically acceptable excipients are described at length by E. W. Martin, in “Remington's Pharmaceutical Sciences”, among others well-known in the art.

A “pharmaceutically acceptable salt” of the present compound generally refers to pharmaceutically acceptable salts form of a compound which can form a salt, because of the existence of for example, amine groups, carboxylic acid groups or other groups which can be ionized in a sample acid-base reaction. A pharmaceutically acceptable salt of an amine compound, such as those contemplated in the current invention, include, for example, ammonium salts having as counterion an inorganic anion such as chloride, bromide, iodide, sulfate, sulfite, nitrate, nitrite, phosphate, and the like, or an organic anion such as acetate, malonate, pyruvate, propionate, fumarate, cinnamate, tosylate, and the like. Certain compounds according to the present invention which have carboxylic acid groups may also form pharmaceutically acceptable salts, generally, as carboxylate salts.

Aspects of the present invention include compounds which have been described in detail hereinabove or to pharmaceutical compositions which comprise an effective amount of one or more compounds according to the present invention, optionally in combination with a pharmaceutically acceptable carrier, additive or excipient.

Another aspect of the present invention is directed to compounds according to the present invention which may be used to mimic α-helical proteins in an agonistic or antagonistic manner. In this aspect of the present invention, one or more of the compounds according to the present invention may be used to mimic or inhibit the binding of an α-helical protein for its binding site, whether that binding site is another protein, a receptor (such as a cell surface receptor or a G-protein coupled receptor), signaling proteins, proteins involved in apoptotic pathways (especially neuronal apoptosis), active sites and regulatory domains of enzymes, growth factors, DNA, RNA (including oligonucleotides), viral fusion proteins and viral coat proteins, among numerous others. In certain aspects of the present invention, one or more compound according to the present invention may be used to inhibit the binding of calmodulin to a calmodulin dependent phosphodiesterase enzyme (PDE1A, PDE1B or PDE1C).

In another aspect, the present invention is directed to the use of one or more compounds according to the present invention in a pharmaceutically acceptable carrier, additive or excipient at a suitable dose ranging from about 0.05 to about 100 mg/kg of body weight per day, preferably within the range of about 0.1 to 50 mg/kg/day, most preferably in the range of 1 to 20 mg/kg/day. The desired dose may conveniently be presented in a single dose or as divided doses administered at appropriate intervals, for example as two, three, four or more sub-doses per day.

Ideally, the active ingredient should be administered to achieve effective peak plasma concentrations of the active compound within the range of from about 0.05 to about 5 uM. This may be achieved, for example, by the intravenous injection of about a 0.05 to 10% solution of the active ingredient, optionally in saline, or orally administered as a bolus containing about 1 mg to about 5 g, preferably about 5 mg to about 500 mg of the active ingredient, depending upon the active compound and its intended target. Desirable blood levels may be maintained by a continuous infusion to preferably provide about 0.01 to about 2.0 mg/kg/hour or by intermittent infusions containing about 0.05 to about 15 mg/kg of the active ingredient. Oral dosages, where applicable, will depend on the bioavailability of the compounds from the GI tract, as well as the pharmacokinetics of the compounds to be administered. While it is possible that, for use in therapy, a compound of the invention may be administered as the raw chemical, it is preferable to present the active ingredient as a pharmaceutical formulation, presented in combination with a pharmaceutically acceptable carrier, excipient or additive.

Pharmaceutical formulations include those suitable for oral, rectal, nasal, topical (including buccal and sub-lingual), vaginal or parenteral (including intramuscular, sub-cutaneous and intravenous) administration. Compositions according to the present invention may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives. When desired, the above described formulations may be adapted to provide sustained release characteristics of the active ingredient(s) in the composition using standard methods well-known in the art.

In the pharmaceutical aspect according to the present invention, the compound(s) according to the present invention is formulated preferably in admixture with a pharmaceutically acceptable carrier. In general, it is preferable to administer the pharmaceutical composition orally, but certain formulations may be preferably administered parenterally and in particular, in intravenous or intramuscular dosage form, as well as via other parenteral routes, such as transdermal, buccal, subcutaneous, suppository or other route, including via inhalationo intranasally. Oral dosage forms are preferably administered in tablet or capsule (preferably, hard or soft gelatin) form. Intravenous and intramuscular formulations are preferably administered in sterile saline. Of course, one of ordinary skill in the art may modify the formulations within the teachings of the specification to provide numerous formulations for a particular route of administration without rendering the compositions of the present invention unstable or compromising their therapeutic activity.

In particular, the modification of the present compounds to render them more soluble in water or other vehicle, for example, may be easily accomplished by minor modifications (such as salt formulation, etc.) which are well within the ordinary skill in the art. It is also well within the routineer's skill to modify the route of administration and dosage regimen of a particular compound in order to manage the pharmacokinetics of the present compounds for maximum beneficial effect to the patient.

Formulations containing the compounds of the invention may take the form of solid, semi-solid, lyophilized powder, or liquid dosage forms, such as, for example, tablets, capsules, powders, sustained-release formulations, solutions, suspensions, emulsions, sup-positories, creams, ointments, lotions, aerosols or the like, preferably in unit dosage forms suitable for simple administration of precise dosages.

The compositions typically include a conventional pharmaceutical carrier or excipient and may additionally include other medicinal agents, carriers, and the like. Preferably, the composition will be about 0.05% to about 75-80% by weight of a compound or compounds of the invention, with the remainder consisting of suitable pharmaceutical additives, carriers and/or excipients. For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

Liquid compositions can be prepared by dissolving or dispersing the compounds (about 0.5% to about 20%), and optional pharmaceutical additives, in a carrier, such as, for example, aqueous saline, aqueous dextrose, glycerol, or ethanol, to form a solution or suspension. For use in oral liquid preparation, the composition may be prepared as a solution, suspension, emulsion, or syrup, being supplied either in liquid form or a dried form suitable for hydration in water or normal saline.

When the composition is employed in the form of solid preparations for oral administration, the preparations may be tablets, granules, powders, capsules or the like. In a tablet formulation, the composition is typically formulated with additives, e.g. an excipient such as a saccharide or cellulose preparation, a binder such as starch paste or methyl cellulose, a filler, a disintegrator, and other additives typically used in the manufacture of medical preparations.

An injectable composition for parenteral administration will typically contain the compound in a suitable i.v. solution, such as sterile physiological salt solution. The composition may also be formulated as a suspension in a lipid or phospholipid, in a liposomal suspension, or in an aqueous emulsion.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Methods for preparing such dosage forms are known or will be apparent to those skilled in the art; for example, see “Remington's Pharmaceutical Sciences” (17th Ed., Mack Pub. Co, 1985). The person of ordinary skill will take advantage of favorable pharmacokinetic parameters of the pro-drug forms of the present invention, where applicable, in delivering the present compounds to a patient suffering from a viral infection to maximize the intended effect of the compound.

The pharmaceutical compositions according to the invention may also contain other active ingredients such as antimicrobial agents, antinfective agents, anti-cancer agents or preservatives. Effective amounts or concentrations of each of the active compounds are to be included within the pharmaceutical compositions according to the present invention.

The individual components of such combinations may be administered either sequentially or simultaneously in separate or combined pharmaceutical formulations.

When one or more of the compounds according to the present invention is used in combination with a second therapeutic agent active the dose of each compound may be either the same as or differ from that when the compound is used alone. Appropriate doses will be readily appreciated by those skilled in the art.

In method aspects according to the present invention, one or more pharmaceutical compositions according to the present invention may be administered in the treatment or prevention of any disease state or condition which is modulated by the interaction of an α-helical protein with binding sites for the α-helical protein. Methods for treating conditions or disease states which are modulated through the binding of an α-helical protein according to the present invention comprise administering to a patient in need thereof an effective amount of a compound according to the present invention in an amount and for a duration to treat, resolve, reduce or eliminate the condition or disease state. Conditions or disease states which may be treated using compounds according to the present invention include, for example, viral infections (including Hepatitis B virus (HBV) infections, human immunodeficiency virus (HIV) infections or conditions associated with such infections (AIDS), Herpes Simplex virus infections (HSV) infections, tumors and/or cancer, proliferative diseases including psoriasis, genital warts and hyperproliferative keratinocyte diseases including hyperkeratosis, ichthyosis, keratoderma, lichen planus, hypertension, neuronal disorders so as to promote neuromodulation, asthma, autoimmune diseases including lupus (lupus erythematosus), multiple sclerosis, arthritis, including rheumatoid arthritis, rheumatic diseases, fibromyalgia, Sjögren's disease and Grave's disease, neuronal disorders such as ADD, memory loss, learning and language disorders, and neurodegenerative diseases including Alzheimer's disease and Parkinson's disease, among others.

Compositions according to the present invention may be coadministered with another active compound such as antimicrobial agents, antinfective agents, anti-cancer agents or preservatives. When co-administered with compounds according to the present invention for the treatment of tumors, including cancer, other agents such as antimetabolites, Ara C, etoposide, doxorubicin, taxol, hydroxyurea, vincristine, cytoxan (cyclophosphamide) or mitomycin C, among numerous others, including topoisomerase I and topoisomerase II inhibitors, such as adriamycin, topotecan, campothecin and irinotecan, other agent such as gemcitabine and agents based upon campothecin and cis-platin may be included. These compounds may also be included in pharmaceutical formulations or coadministered with compounds according to the present invention top produce additive or synergistic anti-cancer activity.

The individual components of such combinations as described above may be administered either sequentially or simultaneously in separate or combined pharmaceutical formulations. When one or more of the compounds according to the present invention is used in combination with a second therapeutic agent active the dose of each compound may be either the same as or differ from that when the compound is used alone. Appropriate doses will be readily appreciated by those skilled in the art.

Chemical Synthesis of Terephthalamide Derivatives

Compositions according to the present invention are synthesized using the general and synthetic methods which are set forth below.

General Method

The invention relates to a method for the formation of synthetic pharmaceutically active agents that are mimics of α-helix structure and function. The general chemistry is established through derivation of a terephthalamide by substitution on the phenyl ring and then substituting on each of the two amide groups at the 1 and 4 positions of the pheny moiety of terephthalamide.

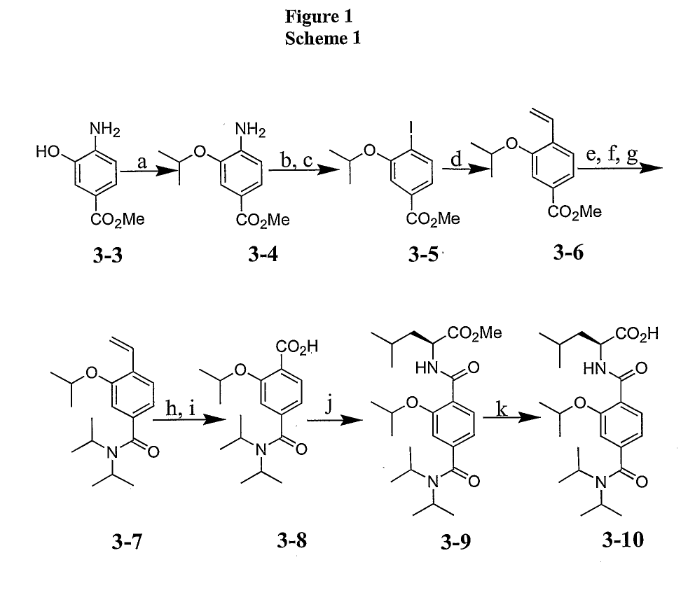

A modular synthesis of terephthalamide derivative 3-10 is shown in FIG. 1, Scheme 1. The 2-isopropoxy group was introduced by O-alkylation. Sandmeyer reaction was used to introduce the iodo-substituent in 3-5. A vinyl group was installed through Stille coupling, followed by hydrolysis of the methyl ester to generate a carboxylic acid at the 4-position. The lower amide bond formation was accomplished using diisopropyl amine to attack the corresponding acid chloride intermediate to afford 3-7. The 1-vinyl group was turned to a carboxylic acid by Lemieux-Johnson and Corey-Schmidt oxidation. Terephthalamide 3-9 was obtained by using standard peptide coupling of 3-8 and L-leucine. This synthesis may be used to generalize the introduction of substituents X and substituents on the two amide groups of the present compounds.

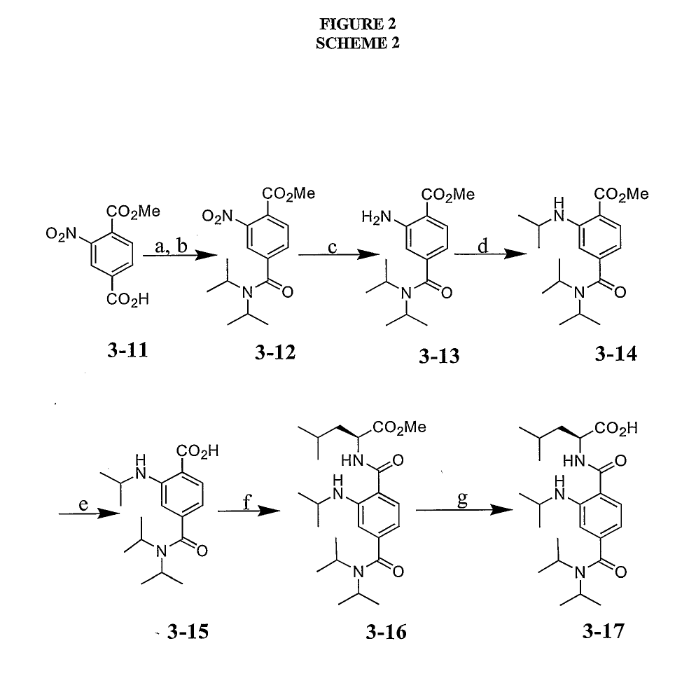

The 2-isopropylamino analogue 3-17 was prepared in a similar fashion (FIG. 2, Scheme 2). Commercially available 2-nitroterephthalic acid-1-methyl ester (3-11) was treated with oxalyl chloride and diispropyl amine to generate N,N-diisopropyl-terephthalamic acid methyl ester (3-12). The 2-nitro group in 3-12 was reduced to the amine group using SnCl₂, then 3-13 was alkylated by means of a reductive amination. Hydrolysis of the methyl ester afforded carboxylic acid 3-15, which was coupled with L-leucine to generate terephthalamide 3-16. Analogous derivatives may be readily synthesized following this general method.

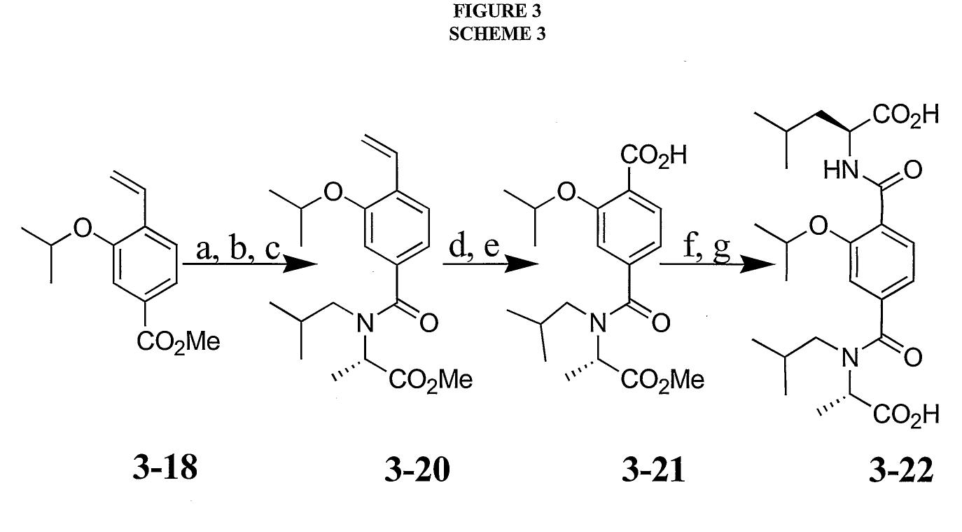

The synthesis of terephthalamides with different substituents on the lower tertiary amide is shown in FIG. 3, Scheme 3. The reactant in step c, asymmetric secondary amine 3-19 (2-isobutylamino-propionic acid methyl ester) was prepared by means of reductive amination of isobutyraldehyde with L-alanine methyl ester.

One of ordinary skill in the art will readily recognize the variations and modifications which can be made to the general synthetic methods which have been presented above.

### EXAMPLES

The following examples illustrate but are not intended in any way to limit the invention.

General. All chemicals were obtained from Sigma-Aldrich, Lancaster or Strem. Mirus LT-1 transfection reagent was purchased from Mirus Corporation (Madison, Wis.). The anti-flag antibody (M2 mouse monoclonal) was purchased from Sigma-Aldrich. The glutathione sepharose 4B beads were purchased from Amersham Biosciences (Piscataway, N.J.). KOAc was dried in oven before use. All solvents were appropriately distilled and all reactions were run under an inert (N2) atmosphere unless otherwise noted. Column chromatography was performed using silica gel (230-400 mesh). ¹H NMR and ¹³C NMR spectra were recorded either on Bruker Avance DPX-500 and DPX-400 spectrometers at room temperature unless otherwise noted. Chemical shifts are expressed as parts per million using solvent or TMS as the internal standard. All low-resolution mass spectra were obtained using Waters LC-MS Micromass ZQ detector at Yale University. Electrospray ionization (ESI)-MS experiments were conducted with 8 kV acceleration potential, 70 eV electron energy, 100 μA emission current and 200° C. ion source temperature. All high-resolution mass spectra (HRMS) were obtained from the Mass Spectrometry Laboratory at the University of Illinois at Urbana-Champaign on a Micormass Q-Tof Ultima quadrupole time of flight mass spectrometer. Specific rotation values were measured on Perkin Elmer Polarimeter 341 using the wavelength of Na/Hal (589 nm) at 20.0° C. Compounds are numbered primarily as they appear in the FIGS. 1-3.

General Syntheses

4-Amino-3-isopropoxy-benzoic acid methyl ester (3-4). To a solution of 2.045 g (12.23 mmol) methyl 4-amino-3-hydroxybenzoate (3-3) in 40 ml acetone, 1.83 ml 2-iodopropane (18.34 mmol, 1.5 eq.) and 7.97g (24.45 mmol, 2.0 eq.) Cs₂CO₃ were added. The resulting mixture was refluxed for 10 h and 10 ml of concentrated ammonium hydroxide was added. The solution was refluxed for 30 min and then cooled to room temperature. The mixture was diluted with water and extracted with Et₂O. The organic layers were combined and washed with brine then dried with MgSO₄. The crude mixture was purified by column chromatography on silica gel (hexane/EtOAc=4/1) to yield 3-4 as a colorless oil (2.300g, 90%). ¹H-NMR (400 MHz, CDCl₃): δ=1.31 (d, 6H, J=6.07 Hz, 2CH₃), 3.82 (s, 3H, CH₃), 4.32 (s, 2H, NH₂), 4.57 (m, 1H, CH), 6.62 (d, 1H, J=8.17 Hz), 7.44 (d, 1H, J=1.71 Hz), 7.50 (dd, 1H, J₁=8.21 Hz, J₂=1.80 Hz). ¹³C-NMR (100 MHz, CDCl₃): δ=21.87, 51.43, 70.47, 113.10, 113.68, 118.81, 123.70, 142.22, 143.81, 167.23. HRMS (ESI) m/z: calcd. for C₁₁H₁₆NO₃ 210.1101, found 210.2032 (M⁺+H).

4-Iodo-3-isopropoxy-benzoic acid methyl ester (3-5). To a solution of 0.75 ml H₂SO₄ (96% in water) in 30 ml H₂O at 0° C., a solution of 2.300 g (11.00 mmol) 3-4 in 20 ml MeOH was added. To the mixture 0.93 g NaNO₂ (13.45 mmol, 1.2 eq.) in 20 ml water was added dropwise over a 20 min period and then the solution was stirred at 0° C. for 15 min. To the resulting mixture, 3.66 g KI (22.01 mmol, 2 eq.) and 63.54 mg Cu (bronze) (1.00 mmol, 0.1 eq.) were added. The solution was refluxed for 8 h and then cooled to room temperature. MeOH was removed at reduced pressure. The mixture was extracted with CH₂Cl₂. The organic layers were combined and washed with a saturated Na₂S₂O₃ (aq.) solution and brine and then dried with MgSO₄. The crude material was purified by column chromatography on silica gel (hexane/EtOAc=4/1) to yield 3-5 as a yellowish oil (3.037 g, 86%). ¹H-NMR (400 MHz, CDCl₃): δ=1.24 (d, 6H, J=6.14 Hz, 2CH₃), 3.74 (s, 3H, CH₃), 4.48 (m, 1H, CH), 7.12 (dd, 1H, J₁=8.10 Hz, J₂=1.74 Hz), 7.26 (s, 1H), 7.65 (d, 1H, J=8.11 Hz). ¹³C-NMR (100 MHz, CDCl₃): δ=21.35, 51.60, 71.27, 94.37, 113.14, 122.31, 130.63, 138.82, 155.98, 165.35. HRMS (ESI) m/z: calcd. for C₁₁H₁₄IO₃ 321.1325, found 321.1330 (M⁺+H).

3-Isopropoxy-4-vinyl-benzoic acid methyl ester (3-6). To a solution of 3.037 g 3-5 (9.49 mmol) and 877 mg Pd(PPh₃)₄ (0.760 mmol, 0.08 eq.) in 20 ml deoxygenated anhydrous toluene, 3.33 ml tributyl(vinyl)tin (11.40 mmol, 1.2 eq.) was added. The solution was refluxed for 12 h and then cooled to room temperature. The solution was concentrated at reduced pressure. Water was added and the mixture was extracted with CH₂Cl₂. The organic layers were combined and then dried with MgSO₄. The crude material was purified by column chromatography on silica gel (hexane/EtOAc=9/1 to 4/1) to yield 3-6 as a clear yellow oil (2.001 g, 95%). ¹H-NMR (400 MHz, CDCl₃): δ=1.20 (d, 6H, J=5.99 Hz, 2CH₃), 3.65 (s, 3H, CH₃), 4.38 (m, 1H, CH), 5.12 (d, 1H, J=11.18 Hz), 5.63 (d, 1H, J=17.79 Hz), 6.89 (q, 1H), 7.28 (d, 1H, J=8.27 Hz), 7.35 (s, 1H). ¹³C-NMR (100 MHz, CDCl₃): δ=21.12, 50.98, 69.81, 113.59, 115.19, 120.95, 125.42, 129.45, 130.55, 131.26, 154.05, 165.46. HRMS (ESI) m/z: calcd. for C₁₃H₁₇O₃ 221.2084, found 221.4115 (M⁺+H).

3-Isopropoxy-N,N-diisopropyl-4-vinyl-benzamide (3-7). To a solution of 2.001 g 3-6 (9.11 mmol) in 30 ml MeOH, 30 ml 6M NaOH (aq.) (18.00 mmol, 2 eq.) was added. The solution was stirred at room temperature overnight then concentrated under reduced pressure. Water was added to the mixture, which was then acidified to pH=1. EtOAc was used to extract the product into the organic layer. The combined organic layers were washed with brine and then dried with MgSO₄. EtOAc was removed to afford the crude 3-isopropoxy-4-vinyl-benzoic acid, which was ready for the next step. ¹H-NMR (400 MHz, CDCl₃): δ=1.24 (d, 6H, J=6.10 Hz, 2CH₃), 5.22 (d, 1H, J=11.21 Hz, CH), 5.72 (d, 1H, J=17.80 Hz), 6.95 (q, 1H), 7.39 (d, 1H, J=8.06 Hz), 7.48 (s, 1H), 7.53 (dd, 1H, J, =7.96 Hz, J₂=1.28 Hz), 12.59 (s, 1H, CO₂H). ¹³C-NMR (100 MHz, CDCl₃): δ=21.83, 70.73, 114.77, 116.55, 122.32, 126.20, 129.12, 131.072, 132.97, 154.71, 172.182.

3-Isopropoxy-4-vinyl-benzoic acid from the previous step was dissolved in newly distilled CH₂Cl₂ then 1.66 ml oxalyl chloride (18.98 mmol, 2.0 eq.) was slowly added. Drops of DMF were added and the mixture was stirred under room temperature for 2 h. Meanwhile the reaction mixture changed from a suspension to a clear solution. CH₂Cl₂ was removed at low pressure and the crude material was kept under low pressure overnight. To the solution of 3-isopropoxy-4-vinyl-benzoic acid chloride in anhydrous CH₂Cl₂ at 0° C., 3.32 ml N,N-diisopropylamine (23.73 mmol, 2.5 eq.) was added slowly and the resulting mixture was stirred under room temperature for 3h. The solution was washed with water and column chromatography on silica get (hexane/EtOAc=7/3) was used to purify the product to yield 3-7 as a colorless, clear oil (2.037 g, 77% after 2 steps). ¹H-NMR (400 MHz, CDCl₃): δ=1.00 (s, 6H, 2CH₃), 1.16 (d, 6H, J=6.13 Hz, 2CH₃), 3.09 (s, 2H, CH₂), 3.32 (s, 2H, CH₂), 4.39 (m, 1H, CH), 5.08 (d, 1H, J=11.30 Hz, CH), 5.60 (d, 1H, J=17.85 Hz, CH), 6.74 (d, 1H, J=8.66 Hz), 6.76 (s, 1H), 6.90 (q, 1H), 7.20 (d, 1H, J=7.78 Hz). ¹³C-NMR (125 MHz, CDCl₃): δ=20.88, 69.58, 110.90, 113.54, 117.22, 125.19, 127.27, 130.15, 136.48, 153.78, 169.38. HRMS (ESI) m/z: calcd. for C₁₈H₂₈NO₂ 290.2433, found 290.2437 (M⁺+H).

2-Isopropoxy-N,N-diisopropyl-terephthalamic acid (3-8). To a solution of 347.9 mg 3-7 (1.20 mmol) in 12 ml 2:1 tBuOH/CCl₄ mixture 4 ml water was added. 15.3 mg OSO₄ (0.06 mmol, 0.05 eq.) was added and the mixture was stirred under room temperature for 15 min. 642.8 mg NaIO₄ (3.00 mmol, 2.5 eq.) was added and the suspension was stirred for 4 h. The solution was diluted with water when all the starting material was consumed and the product was extracted into Et₂O. The combined organic layers were washed with 10% NaHSO₃ (aq.) and water then dried with MgSO₄. The solvent was removed under low pressure to yield intermediate 4-formyl-3-isopropoxy-N,N-diisopropyl-benzamide. ¹H-NMR (400 MHz, CDCl₃): δ=1.14 (s, 6H, 2CH₃), 1.39 (d, 6H, J=8.0 Hz, 2CH₃), 1.53 (s, 6H, 2CH₃), 3.51 (s, 1H, NH), 3.76 (s, 1H, NH), 4.70 (m, 1H, CH), 6.88 (d, 1H, J=8.0 Hz), 6.90 (s, 1H), 7.82 (d, 1H, J=8.0 Hz), 10.46 (s, H, CHO).

To a solution of 4-formyl-3-isopropoxy-N,N-diisopropyl-benzamide in DMF, 678.31 mg pyridinium dichromate (1.80 mmol, 1.5 eq.) was added and the resulting mixture was stirred under room temperature for 6 h. Ethyl ether was added. The ether layer was decanted and was extracted with 1M NaOH (aq.). The aqueous layers were combined and acidified to pH=1. The product was then extracted into EtOAc and the organic layer was dried with MgSO₄ to yield 3-8 as a white solid (187.3 mg, 61% after 2 steps). ¹H-NMR (400 MHz, CDCl₃): δ=1.25 (d, 6H, J=8.0 Hz, 2CH₃), 4.56 (m, 1H, CH), 5.26 (d, 1H, J=12.0 Hz), 5.75 (d, 1H, J=12.0 Hz), 6.87 (d, 1H, J=8.0 Hz), 7.03 (m, 1H), 7.27 (s, 1H), 7.46 (d, 1H, J=8.0 Hz). HRMS (ESI) m/z: calcd. for C₁₇H₂₆NO₄ 308.4412, found 308.4410 (M⁺+H).

(S)-2-(4-Diisopropylcarbamoyl-2-isopropoxy-benzoylamino)-4-methyl-pentanoic acid methyl ester (3-9). To a solution of 90.7 mg 3-8 (0.295 mmol), 59.04 mg L-leucine methyl ester hydrochloride (0.325 mmol, 1.1 eq.), 43.92 mg 1-hydroxybenzotriazole (0.325 mmol, 1.1 eq.), 0.05 ml Et₃N (0.325 mmol, 1.1 eq.) in 10 ml anhydrous CH₂Cl₂ at 0° C. and 56.6 mg 1-(3-dimethylaminopropyl)-3-ethylcarbondiimide hydrochloride (0.295 mmol, 1.0 eq.) were added and the resulting mixture was stirred under room temperature for 24 h. The solution was diluted with CH₂Cl₂, and then washed with saturated NaHCO₃ (aq.) solution, 10% citric acid (aq.) solution and water. The solution was dried over MgSO₄ and was in turn concentrated at low pressure. The crude material was purified with column chromatography on silica gel (hexane/EtOAc=1/1) to yield 3-9 as a yellowish oil (159.7 mg, 85%). [α]_{D}=−129.9° (c 0.0283, 20° C., CHCl₃). ¹H-NMR (500 MHz, CDCl₃): δ=0.97 (d, 6H, J=4.0 Hz, 2CH₃), 1.12 (s, 6H, 2CH₃), 1.46 (d, 6H, J=8.0 Hz, 2CH₃), 1.46 (s, 6H, 2CH₃), 1.65 (m, 1H, CH), 1.75 (m, 2H, CH₂), 3.51 (s, 1H, CH), 3.69 (s, 3H, CH₃), 3.72 (s, 1H, CH), 4.81 (m, 2H, 2CH), 6.93 (s, 1H), 6.94 (s, 1H, J=8.0 Hz), 8.19 (d, 1H, J=8.0 Hz), 8.57 (d, 1H, J=8.0 Hz, NH). ¹³C-NMR (100 MHz, MeOH-d₄): δ=21.39, 21.41, 22.82, 22.90, 22.96, 23.89, 26.87, 42.98, 53.40, 53.52, 72.25, 74.28, 113.22, 119.31, 133.74, 133.76, 144.81. HRMS (ESI) m/z: calcd. for C₂₄H₃₉N₂O₅ 435.2859, found 435.2872 (M⁺+H).

(S)-2-(4-Diisopropylcarbamoyl-2-isopropylamino-benzoylamino)-4-methyl-pentanoic acid (3-17). [α]_{D}=−15.6° (c 0.090, 20° C., CHCl₃). ¹H-NMR (400 MHz, MeOH-d₄): δ=0.96 (q, 6H, 2CH₃), 0.15 (s, 6H, 2CH₃), 1.21 (d, 6H, J=6.4 Hz, 2CH₃), 1.52 (s, 6H, 2CH₃), 1.75 (m, 3H, CH₂, CH), 3.65 (m, 2H, NH, CH), 3.87 (s, 1H, CH), 4.58 (m, 1H, CH), 4.91 (s, 1H, OH), 6.48 (d, 1H, J=8.4 Hz), 6.57 (s, 1H), 7.58 (d, 1H, J=8.4 Hz). ¹³C-NMR (100 MHz, MeOH-d₄): δ=20.61, 20.72, 21.59, 22.63, 22.67, 23.28, 26.10, 41.04, 44.73, 46.99, 47.02, 52.09, 52.62, 109.40, 112.21, 117.40, 130.28, 130.33. 130.42, 143.27, 143.29. HRMS (ESI) m/z: calcd. for C₂₃H₃₈N₃O₄ 420.2862, found 419.2865 (M⁺+H).

(S)-2-{4-[Isobutyl-((S)-1-methoxycarbonyl-ethyl)-carbamoyl]-2-isopropoxy-benzoylamino}-4-methyl-pentanoic acid methyl ester (3-22). [α]_{D}=−39.1° (c 0.098, 20° C., CHCl₃). ¹H-NMR (400 MHz, MeOH-d₄): δ=0.72 (d, 6H, J=4.4 Hz, 2CH₃),0.89 (d, 6H, J=8.0 Hz, 2CH₃), 1.34 (d, 6H, J=4.4 Hz, 2CH₃), 1.48 (d, 3H, J=4.0 Hz, CH₃), 1.69 (m, 3H, CH, CH₂), 3.03 (m, 1H, NH), 3.20 (m, 1H), 3.72 (s, 3H, CO₂CH₃), 3.73 (s, 3H, CO₂CH₃), 4.31 (m, 1H, OCH(Me)₂), 4.62 (d, 1H, J=4.0 Hz, NH), 4.85 (t, 1H, CH(iBu)CO₂Me), 6.97 (d, J=8.0 Hz. 1H), 7.04 (s, 1H), 7.86 (d, J=8.0 Hz. 1H), 8.39 (s, 1H, NH). ¹³C-NMR (100 MHz, MeOH-d₄): δ=14.42, 19.89, 20.00, 21.78, 21.81, 21.86, 22.83, 25.72, 28.25, 41.85, 41.89, 57.19, 58.92, 73.32, 113.57, 119.58, 132.30, 141.50, 157.02, 166.70, 172.11, 172.75, 174.11. HRMS (ESI) m/z: calcd. for C₂₆H₄₁N₂O₇ 493.2914, found 493.2907 (M⁺+H).

(S)-2-(4-Diisopropylcarbamoyl-2-methoxy-benzoylamino)-4-methyl-pentanoic acid methyl ester (3-26). [α]_{D}=−4.77° (0.0125, 20° C., CHCl₃). ¹H-NMR (400 MHz, CDCl₃): δ=0.96, (dd, 6H, J₁=8.0 Hz, J₂=4.0 Hz, 2CH₃), 1.12 (broad, 6H, NCH(CH₃)₂), 1.51 (broad, 6H, NCH(CH₃)₂), 1.68 (m, 3H, CH,CH₂), 3.50 (broad, 2H, 2NCH(CH₃)₂), 3.73 (s, 3H, CO₂CH₃), 3.99 (t, 3H, OCH₃), 4.80 (m, 1H, CHCO₂Me), 6.93 (s, 1H), 6.95 (dd, 1H, J₁=7.88 Hz, J₂=1.36 Hz), 8.16 (d, 1H, J=7.88 Hz), 8.26 (d, 1H, J=7.56 Hz, NH). ¹³C-NMR (100 MHz, MeOH-d₄): δ=21.90, 22.48, 23.68, 26.63, 42.18, 47.75, 48.77, 53.21, 53.27, 57.33, 110.67, 119.08, 124.01, 132.97, 144.44, 159.75, 167.90, 172.47, 174.98. HRMS (ESI) m/z: calcd. for C₂₂H₃₅N₂O₅ 407.2546, found 407.2547 (M⁺+H).

(S)-2-(4-Dimethylcarbamoyl-2-isopropoxy-benzoylamino)-propionic acid methyl ester (3-33). [α]_{D}=+7.04° (c 0.0018, 20° C., CHCl₃). ¹H-NMR (400 MHz, MeOH-d₄): δ=1.50 (m, 4H, CH₃, CH), 1.59 (s, 6H, 2CH₃), 2.96 (s, 3H, NCH₃), 3.11 (s, 3H, NCH₃), 3.77 (s, 3H, CO₂CH₃), 4.82 (m, 2H, NH, OCH(CH₃)₂), 7.03 (d, 1H, J=8.00 Hz), 7.06 (s, 1H), 8.21 (d, 1H, J=8.00 Hz), 8.79 (d, 1H, J=6.00 Hz, NH). ¹³C-NMR (100 MHz, CDCl₃): δ=19.17, 22.39, 22.52, 49.03, 52.82, 72.88, 113.21, 119.64, 132, 84, 156.68, 174.05. HRMS (ESI) m/z: calcd. for C₁₇H₂₅N₂O₅ 337.1763, found 337.1766 (M⁺+H).

(S)-2-(4-Dimethylcarbamoyl-2-isopropoxy-benzoylamino)-3-phenyl-propionic acid (3-35). [α]_{D}=+57.8° (c 0.008, 20° C., CHCl₃). ¹H-NMR (500 MHz, CDCl₃): δ=2.89 (s, 3H, NCH₃), 3.01 (s, 3H, NCH₃), 3.30 (m, 2H, CH₂), 4.72 (m, 2H, NH, OCH(CH₃)₂), 5.10 (broad, 1H, COOH), 6.96 (d, 1H, J=8.00 Hz), 7.11 (s, 1H), 8.25 (d, 1H, J=8.00 Hz), 8.72 (d, 1H, J=7.50 Hz, NH). ¹³C-NMR (125 MHz, CDCl₃): δ=21.19, 21.28, 21.73, 37.00, 53.62, 72.02, 114.68, 118.85, 121.66, 125.20, 126.76, 126.80, 129.19, 132.28, 133.08, 135.65, 155.77, 164.06, 169.53, 174.89. HRMS (ESI) m/z: calcd. for C₂₂H₂₆N₂O₅ 399.1920, found 399.1935 (M⁺+H).

(S)-2-(4-Diethylcarbamoyl-2-isopropoxy-benzoylamino)-propionic acid methyl ester (3-37). [α]_{D}=+22.2° (c 0.0176, 20° C., CHCl₃). ¹H-NMR (400 MHz, CDCl₃): δ=1.03 (s, 3H, CH₃), 1.18 (s, 3H, CH₃), 1.42 (m, 9H, 3CH₃), 3.16 (s, 2H, CH₂), 3.47 (s, 2H, CH₂), 3.71 (s, 3H, CH₃), 4.72 (m, 2H, 2CH), 6.93 (s, 1H), 6.94 (s, 1H), 8.14 (d, 1H, J=8.29 Hz), 8.71 (d, 1H, J=6.79 Hz, NH). ¹³C-NMR (125 MHz, CDCl₃): δ=12.748, 14.132, 18.596, 21.829, 39.207, 43.087, 48.463, 52.243, 72.317, 111.921, 118.339, 122.171, 132.394, 141.344, 156.116, 163.940, 169.909, 173.438. HRMS (ESI) m/z: calcd. for C₁₉H₂₉N₂O₅ 365.2076, found 365.2075 (M⁺+H).

(S)-2-(4-Diethylcarbamoyl-2-isopropoxy-benzoylamino)-4-methyl-pentanoic acid methyl ester (3-38). [α]_{D}=+11.2° (c 0.0189, 20° C., CHCl₃). ¹H-NMR (400 MHz, CDCl₃): δ=0.91 (d, 6H, J=6.15 Hz, 2CH₃), 1.03 (s, 3H, CH₃), 1.17 (s, 3H, CH₃), 1.40 (m, 6H, 2CH₃), 1.68 (m, 2H, CH and CH₂), 3.17 (s, 2H, CH₂), 3.48 (s, 2H, CH₂), 3.69 (s, 3H, CH₃), 4.48 (m, 2H, 2CH), 6.92 (s, 1H), 6.94 (s, 1H), 8.13 (d, 1H, J=8.34 Hz), 8.53 (d, 1H, J=7.64 Hz, NH). ¹³C-NMR (125 MHz, CDCl₃): δ=21.900, 22.083, 22.702, 24.910, 41.762, 51.126, 72.205, 111.917, 118.436, 122.191, 132.563, 141.401, 156.099, 164.285, 169.957, 173.455. HRMS (ESI) m/z: calcd. for C₂₂H₃₅N₂O₅ 407.2546, found 407.2551 (M⁺+H).

(S)-2-(4-Diisobutylcarbamoyl-2isopropoxy-benzoylamino)-propionic acid methyl ester (3-39). [α]_{D}=+15.2° (c 0.0122, 20° C., CHCl₃). ¹H-NMR (400 MHz, CDCl₃): δ=0.67 (d, 6H, J=6.41 Hz, 2CH₃), 0.92 (d, 6H, J=6.50 Hz, 2CH₃), 1.40 (m, 6H, 2CH₃), 1.45 (d, 3H, J=7.10 Hz, CH₃), 1.78 (m, 1H, CH), 2.05 (m, 1H, CH), 3.01 (d, 2H, J=7.39 Hz, CH₂), 3.28 (m, 2H, CH₂), 3.72 (s, 3H, CH₃), 4.73 (m, 2H, 2CH), 6.89 (s, 1H), 6.92 (s, 1H), 8.14 (d, 1H, J=7.90 Hz), 8.72 (d, 1H, J=6.76 Hz, NH). ¹³C-NMR (125 MHz, CDCl₃): δ=18.648, 19.690, 20.104, 21.812, 21.957, 26.065, 26.650, 48.540, 50.993, 52.314, 56.315, 72.335, 112.893, 119.085, 122.173, 132.310, 141.651, 156.183, 164.072, 171.186, 173.550. HRMS (ESI) m/z: calcd. for C₂₃H₃₇N₂O₅ 421.2702, found 421.2714 (M⁺+H).

(S)-2-(4-Diisobutylcarbamoyl-2-isopropoxy-benzoylamino)-4-methyl-pentanoic acid methyl ester (3-40). [α]_{D}=+8.28° (c 0.0174, 20° C., CHCl₃). ¹H-NMR (400 MHz, CDCl₃): δ=0.68 (d, 6H, J=5.99 Hz, 2CH₃), 0.92 (m, 6H, 2CH₃), 1.40 (m, 6H, 2CH₃), 1.68 (m, 3H, CH₂ and CH), 1.79 (m, 1H, CH), 2.05 (m, 1H, CH), 3.01 (d, 2H, J=7.41 Hz CH₂), 3.28 (m, 2H, CH₂), 3.70 (s, 3H, CH₃), 4.76 (m, 2H, 2CH), 6.89 (d, 1H, J=7.96 Hz), 6.92 (s, 1H), 8.14 (d, 1H, J=7.90 Hz), 8.54 (d, 1H, J=7.63 Hz, NH). ¹³C-NMR (125 MHz, CDCl₃): δ=19.702, 20.105, 21.827, 21.886, 21.997, 22.093, 22.714, 24.921, 26.069, 26.651, 41.774, 50.980, 51.250, 52.150, 56.325, 72.167, 112.828, 119.108, 122.130, 132.407, 141.659, 156.110, 164.357, 171.161, 173.498. HRMS (ESI) m/z: calcd. for C₂₆H₄₃N₂O₅ 463,3172, found 463.3175 (M⁺+H).

(S)-2-(4-Diisobutylcarbamoyl-2-isopropoxy-benzoylamino)-3-methyl-butyric acid methyl ester (3-41). [α]_{D}=+20.6° (c 0.0158, 20° C., CHCl₃). ¹H-NMR (400 MHz, CDCl₃): δ=0.68 (d, 6H, J=6.26 Hz, 2CH₃), 0.94 (m, 12H, 4CH₃), 1.41 (m, 6H, 2CH₃), 1.78 (m, 1H, CH), 2.05 (m, 1H, CH), 2.22 (m, 1H, CH), 3.02 (d, 2H, J=7.47 Hz, CH₂), 3.30 (m, 2H, CH₂), 3.70 (s, 3H, CH₃), 4.76 (m, 2H, 2CH), 6.90 (d, 1H, J=7.99 Hz), 6.93 (s, 1H), 8.15 (d, 1H, J=7.96 Hz), 8.65 (d, 1H, J=8.40 Hz, NH). ¹³C-NMR (125 MHz, CDCl₃): δ=17.984, 18.968, 19.692, 20.112, 21.781, 22.048, 26.058, 26.642, 31.409, 50.972, 51.957, 56.317, 57.534, 71.916, 112.651, 119.024, 122.157, 132.493, 141.637, 156.114, 164.493, 171.187, 172.422. HRMS (ESI) m/z: calcd. for C₂₅H₄₁N₂O₅ 449.3015, found 449.3018 (M⁺+H).

(S)-2-(4-Diphenylcarbamoyl-2-isopropoxy-benzoylamino)-propionic acid (3-42). [α]_{D}=+41.86° (c 0.071, 20° C., CHCl₃). ¹H-NMR (400 MHz, MeOH-d₄): δ=1.46 (m, 9H, CHCH₃, CH(CH₃)₂), 4.61 (m, 1H, CHCO₂Me), 4.86 (broad, 1H, COOH), 4.94 (m, 1H, OCH(CH₃)₂), 6.81 (d, 1H, J=8.12 Hz, CH), 7.03-7.61 (10H), 7.69 (d, 1H, J=8.12 Hz, CH), 7.74 (s, 1H, CH), 8.06 (d, 1H, J=8.0 Hz, NH). ¹³C-NMR (100 MHz, CDCl₃): δ=17.30, 20.85, 20.92, 20.99, 47.78, 71.52, 114.07, 115.44, 117.85, 118.88, 121.18, 123.21, 124.29, 124.36, 125.34, 126.45, 126.82, 127.92, 128.01, 128.83, 131.45, 133.06, 154.96, 161.03, 163.49, 166.54, 173.34. HRMS (ESI) m/z: calcd. for C₂₆H₂₇N₂O₅ 447.1920, found 447.1926 (M⁺+H).

(S)-2-(4-Diphenylcarbamoyl-2-isopropoxy-benzoylamino)-4-methyl-pentanoic acid (3-43). [α]_{D}=−14.4° (c 0.053, 20° C., CHCl₃). ¹H-NMR (400 MHz, MeOH-d₄): δ=0.98 (m, 6H, CH(CH₃)₂), 1.45 (dd, 6H, J₁=5.19 Hz, J₂=3.6 Hz, 2CH₃), 1.76 (m, 3H, CH, CH₂), 4.69 (m, 1H, CHCO₂Me), 4.92 (m, 1H, OCH(CH₃)₂), 6.81 (d, 1H, J=5.6 Hz, CH), 7.03-7.21 (10H), 7.67 (dd, 1H, J₁=5.6 Hz, J₂=1.6 Hz, CH), 7.74 (d, 1H, J=1.6 Hz, CH), 8.00 (d, 1H, J=8.0 Hz, NH). ¹³C-NMR (125 MHz, CDCl₃): δ=20.11, 23.46, 28.75, 42.26, 53.62, 74.13, 116.91, 122.08, 127.09, 128.25, 128.97, 132.46, 140.53, 141.78, 155.84, 168.49, 172.36, 173.91. HRMS (ESI) m/z: calcd. for C₂₉H₃₃N₂O₅ 489.2389, found 489.2405 (M⁺+H).

(S)-2-(4-Diphenylcarbamoyl-2-isopropoxy-benzoylamino)-4-methyl-pentanoic acid methyl ester (3-44). [α]_{D}=−7.37° (c 0.0033, 20° C., CHCl₃). ¹H-NMR (500 MHz, CDCl₃): δ=0.88 (d, 6H, J=5.8 Hz, 2CHCH₃), 1.36 (dd, 6H, J₁=15.9 Hz, J₂=6.1 Hz, OCH(CH₃)₂), 1.56 (m, 1H, CH(CH₃)₂), 1.58 (m, 2H, CH₂), 3.67 (s, 3H, CO₂CH₃), 4.51 (m, 1H, CHCO₂Me), 4.73 (m, 1H, OCH(CH₃)₂), 6.98-7.57 (12H), 7.95 (d, 1H, J=8.10 Hz, CH), 8.46 (d, 1H, J=7.65 Hz, NH). ¹³C-NMR (125 MHz, CDCl₃): δ=22.20, 22.39, 22.54, 23.11, 25.35, 42.21, 51.70, 52.58, 72.74, 115.35, 122.15, 123.49, 127.09, 127.74, 129.67, 132.46, 140.51, 143.88, 155.88, 164.65, 169.79, 173.92. HRMS (ESI) m/z: calcd. for C₃₀H₃₅N₂O₅ 503.2546, found 503.2528 (M⁺+H).

(S)-2-(4-Diphenylcarbamoyl-2-isopropoxy-benzoylamino)-3-phenyl-propionic acid (3-45). [α9 _{D}=+29.2° (c 0.090, 20° C., CHCl₃). ¹H-NMR (400 MHz, MeOH-d₄): δ=0.93 (dd, 6H, J₁=25.2 Hz, J₂=5.6 Hz, 2CH₃), 4.77 (broad, 1H, COOH), 4.78 (m, 1H, OCH(CH₃)₂), 6.25-8.03 (19H). ¹³C-NMR (125 MHz, CDCl₃): δ=21.33, 21.36, 21.43, 31.02, 31.98, 54.72, 73.10, 115.62, 117.88, 120.64, 122.24, 127.46, 127.50, 128.97, 129.01, 129.63, 129.95, 130.15, 130.19, 132.35, 137.25, 157.03, 165.53, 173.42. HRMS (ESI) m/z: calcd. for C₃₂H₃₁N₂O₅ 523.2233, found 5223.2231 (M⁺+H).

(S)-2-{4-[((S)-1-Carboxy-ethyl)-isobutyl-carbamoyl]-2-(naphthalen-2-yloxy)-benzoylamino}-4-methyl-pentanoic acid (3-50). [α]_{D}=+11.9° (c 0.031, 20° C., CHCl₃). ¹H-NMR (500 MHz, MeOH-d₄): δ=0.68 (dd, 6H, J₁=5.2 Hz, J₂=2.0 Hz, 2CH₃), 0.92 (dd, 6H, J₁=7.6 Hz, J₂=3.6 Hz, 2CH₃), 1.17 (m, 1H), 1.43 (m, 1H), 1.57 (m, 2H, CH₂), 1.71 (m, 2H), 1.76 (m, 1H, CH), 4.10 (m, 1H), 4.58 (m, 2H), 5.10 (broad, s, 2COOH), 7.28-7.92 (10H). ¹³C-NMR (125 MHz, MeOH-d₄): δ=21.17, 21.22, 21.26, 22.69, 22.76, 22.89, 25.37, 25.70, 25.76, 40.68, 41.22, 52.08, 52.28, 61.86, 61.93, 114.54, 114.57, 119.85, 119.91, 123.42, 123.46, 125.74, 127.37, 127.88, 128.35, 130.39, 130.94, 131.38, 131.58, 135.25, 138.89, 155.12, 166.96. HRMS (ESI) m/z: calcd. for C₃₀H₃₅N₂O₇ 535.2444, found 535.2450 (M⁺+H).

Fluorescence polarization assays. Fluorescence polarization experiments were conducted on a Photon Technology International instrument using a 0.3 cm path length cuvette. Spectra were measured at 25° C. using 10.0 nm slit widths. Fluoroscein-labled Bak peptide (F1-GQVGRQLAIIGDDINR-CONH₂) was purchased from the HHMI Biopolymer/Keck Foundation Biotechnology Resource Center at the Yale University School of Medicine (New Haven, Conn.). The N-terminus of the peptide was capped with the fluorophore and the C-terminus was amidated. Bcl-x_{L} was expressed and purified as previously described.^{[7]} Excitation at 495 nm was used for the fluorescein-containing peptide and the excimer emission maximum at 535 nm was monitored. Polarization measurements were recorded upon titration of inhibitors (ca. 10 mM stock solutions in DMSO) at varying concentrations into a solution of 15 nM Fl-Bak and 184 nM Bcl-x_{L} (25° C., 10.0 mM PBS, pH 7.4). Regression analysis was carried out using SigmaPlot 2001 (Systat Co.) ligand binding macro module. Experimental data were fitted into equation (1) to determine the IC₅₀ values, which in turn can be related to the known affinity of the 16-mer Bak peptide (K_{d}=120 nM) to acquire the inhibitory constant Kᵢ.^{[11, 21]}
y=min+(max−min)/(1+10^{x-logIc50}) Equation (1)
(y=total binding, x=log concentration of ligand, min=nonspecific binding, max=maximum binding in absence of ligand)

The binding affinity of the terephthalamide molecules for Bcl-x_{L} was assessed by a previously reported fluorescence polarization assay using a fluorescently labeled 16-mer Bak-peptide (F1-GQVGRQLAIIGDDINR-CONH₂).^{[14]} Displacement of this probe through competitive binding of the terephthalamide into the hydrophobic cleft of Bcl-x_{L} leads to a decrease in its fluorescence polarization. Computational regression analysis was conducted to determine the IC₅₀ values, which in turn can be related to the known affinity of the 16-mer Bak peptide (K_{d}=0.120 μM) to acquire the inhibitory constant (Kᵢ) of the inhibitors.^{[11, 21]} To test the validity of this assay, we used a non-labeled Bak-peptide as the competitive inhibitor to bind Bcl-x_{L}, giving a Kᵢ of 0.122 μM, which closely corresponds to the K_{d} value obtained from the saturation titration experiment.

A series of terephthalamides with varied side chains were prepared as described above and are presented in the following Table 1. All the assays were carried out with 0.01 μM to 1,000 μM terephthalamide solution in 10 mM PBS buffer (pH=7.4, 298K) with less than 0.1% DMSO, a testament to the good solubility of terephthalamides in water. Table 3.1 shows that terephthalamide 3-9 has good affinity for Bcl-x_{L} with a Kᵢ value of 0.78±0.07 μM. By screening compounds with a range of side chains on the upper carboxamide, we found that the isobutyl group as the upper substituent provided the best inhibition results (3-9, 3-10, 3-29, 3-34). The newly introduced stereogenic center in the terephthalamide did not affect the affinity, as seen by comparing 3-26 and 3-27. The optimal alkoxy group in the 2-position of terephthalamide was found to be isopropoxy (3-9, 3-10, 3-34, 3-37), which closely mimics the size of Leu78 of the Bak peptide; both larger (3-29, 3-31) and smaller (3-26, 3-27) substituents gave decreased affinities. The N,N-alkyl substituents on the lower carboxaimide were shown to favor the medium to small substituents since N,N-dimethyl (3-34), -diethyl (3-37), and -diisopropyl (3-9, 3-10) terephthalamide analogues have low micro-molar Kᵢ values while most of the affinity was lost when the alkyl substituents were replaced by phenyl groups (3-42, 3-43, 3-44, 3-45). Comparison of the terephthalamide derivatives with the free amino acid in the upper carboxamide moiety and their methyl esters suggested that the ester group did not significantly affect the binding (3-9, 3-10). However, for less hydrophilic compounds, such as 3-31 and 3-32, only the free acid derivatives are soluble in aqueous solution. The 2-isopropylamino terephthalamide 3-17 showed affinity 4-fold less than its 2-isopropoxy analogue 3-10, suggesting the intramolecular hydrogen bond in 3-10 helps to orient the side chain and in turn to enhance binding. The importance of hydrophobic side chains was further confirmed by the weak binding of 3-36, which lacks the key substituents. The series of terephthalamide derivatives with asymmetric substituents on the lower carboxamide moiety did not show activity in disrupting the Bcl-x_{L}/Bak interaction (3-22, 3-46-3-49, 3-51) with an exception of 3-50, possibly due to a different binding mode. These assay results confirmed that the terephthalamide derivatives retained the high in vitro affinity of the a prior art terphenyl scaffold while reducing the complexity of the chemistry involved.

TABLE 1
Results of the fluorescence polarization competition assay of terephthalamide
derivatives as the antagonists of Bcl-x_{L}/Bak complex.

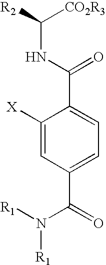

| | | | | | K₁ ± S.D. |
|---|---|---|---|---|---|
| R₁ | X | R₂ | R₃ | Compound | (μM)* |
| -iPr | —OiPr | -iBu | —Me | 3-9 | 0.781 ± 0.070 |
| | | -iBu | —H | 3-10 | 0.839 ± 0.171 |
| | | -Me | -Me | 3-23 | 10.96 ± 1.882 |
| | | -iPr | -Me | 3-24 | 5.846 ± 2.070 |
| | | -Bn | -Me | 3-25 | 10.92 ± 5.170 |
| | —OMe | (S)-iBu | -Me | 3-26 | 1.852 ± 0.318 |
| | | (R)-iBu | -Me | 3-27 | 1.785 ± 0.388 |
| | | (S)-iBu | —H | 3-28 | 2.310 ± 0.329 |
| | —OPh | -iBu | —H | 3-29 | 1.013 ± 0.053 |
| | | -iBu | -Me | 3-30 | Not soluble |
| | —ONaphthlene | -iBu | —H | 3-31 | 7.631 ± 1.021 |
| | —O(p- | -iBu | -Me | 3-32 | 13.15 ±]8.800 |
| | Nitrophenyl) | | | | |
| | —NH(iPr) | -iBu | —H | 3-17 | 3.313 ± 0.345 |
| -Me | —OiPr | -Me | -Me | 3-33 | 8.343 ± 1.606 |
| | | -iBu | -Me | 3-34 | 3.141 ± 0.306 |
| | | —Bn | —H | 3-35 | 8.917 ± 3.593 |
| | H | —H | -Me | 3-36 | No affinity |
| -Et | —OiPr | -Me | -Me | 3-37 | 2.436 ± 0.947 |
| | | -iBu | -Me | 3-38 | 11.44 ± 5.09 |
| -iBu | —OiPr | -Me | -Me | 3-39 | 8.764 ± 2.783 |
| | | -iBu | -Me | 3-40 | 8.364 ± 3.804 |
| | | -iPr | -Me | 3-41 | 138.1 ± 49.5 |
| -Ph | —OiPr | -Me | —H | 3-42 | 260 ± 58 |
| | | -iBu | —H | 3-43 | 780 ± 174 |
| | | -iBu | -Me | 3-44 | No affinity |
| | | -Bn | —H | 3-45 | No affinity |

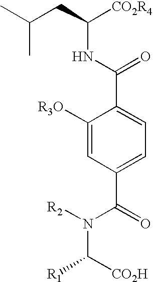

| | | | | | K1+S.D. |
|---|---|---|---|---|---|
| R₁ | R₂ | R₃ | R₄ | Compound | (μM) |
| -Me | -iBu | -iPr | —H | 3-22 | No affinity |
| —H | —H | —H | —H | 3-46 | No affinity |
| -iPr | -iBu | -iPr | —H | 3-47 | 450 ± 231 |
| -iBu | —H | -Ph | —H | 3-48 | No affinity |
| -iBu | —H | -Ph | -Me | 3-49 | Not soluble |
| -iBu | —H | -(2- | —H | 3-50 | 82.1 ± 11 |
| | | Naphthlene) | | | |
| -iBu -H | -(2-Napthlene) | -Me | 3-51 | Not soluble | |

*Each Kᵢ value represents the mean average of three independent experiments.

(¹⁵N, ¹H)-amide chemical shift pretubation experiments.

(¹⁵N, ¹H)-2D-HSQC spectra were recorded on a Varian DPX-600 spectrometer. The concentration of Bcl-x_{L} was 2 mM, with 0, 1 mM, 2 mM 3-26, respectively, in 10% DMSO/D₂O (25° C., 10.0 mM PBS, pH 7.0).

The displacement of Bak peptide by terephthalamide in the fluorescence polarization suggests that the inhibitor and the peptide bind to the same surface area of Bcl-x_{L} protein. To structurally probe the binding mode of the terephthalamide, (¹⁵N, ¹H)-2D-amide chemical shift perturbation mapping with ¹⁵N-labeled Bcl-x_{L} were performed as above. Addition of compound 3-26 led to shifts in a number of residues on the surface of Bcl-x_{L}. The residues of A89, G94, Y101, R102, L108, L112, A119, R139, A142 showed significant chemical shift changes upon the addition of the synthetic inhibitor 3-26. Some other residues, including E96, H113, I114, V127, I140, S145, V161 showed moderate chemical shift change under the same conditions. These affected residues all lie in the shallow cleft on the protein into which the Bak helix binds. An overlay of 3-26 and the Bak peptide within the binding pocket suggests that the terephthalamide is indeed mimicking the cylindrical shape of the helix with the substituents making a series of hydrophobic contacts with the protein. The residues V74, L78, and I81 of Bak BH3, which the terephthalamide has been designed to mimic, are within 4 Å of residues F97, R102, L108, I140, and A142 of Bcl-x_{L}, all of which showed significant chemical shift changes upon addition of helical mimetic 3-26. The effect on F97 was unclear due to overlap with N5 although it seems to shift significantly. These results confirmed that terephthalamide 3-26 targets the same cleft which Bak BH3 peptide recognizes.

Comparison of these results with ¹⁵N, ¹H—HSQC mapping of the previously reported terphenyl 3-1 (R₁=R₃=iBu, R₂=1-naphthlenemethylene) suggested a similar binding mode to Bcl-x_{L} for both proteomimetics.^{11]} The residues mostly affected by both of the inhibitors are G94 in BH3, R102 and 108 in the C-terminal region of BH2, and R139, I140 and A142 in BH1 domain of Bcl-x_{L}. These residues are all found in the conserved hydrophobic pocket on Bcl-x_{L} where the Bak BH3 helix binds.^{[14]} The chemical shift perturbation of these or their neighboring residues listed above indicated that both inhibitors and the Bak BH3 peptide target the same area on the Bcl-x_{L} exterior surface, suggesting terephthalamide is a successful alternative scaffold to terphenyl as a mimetic of α-helices.

Docking studies. The docking studies were performed using AutoDock 3.0.^{[22]} A genetic algorithm (Lamarckian Genetic Algorithm, or LGA for short) was used and the torsion angles of the ligand were varied using AUTOTORS. All other procedures for the docking experiment were followed as described in the users manual for the AUTODOCK program.

Docked conformations were ranked automatically by the AUTODOCK program using a force field scoring function. A total of 100 distinct conformational clusters were found out of 100 runs using an rmsd-tolerance of 1.0 Å. Among those, one of the highest ranked docked structures was used for molecular visualization.

The results of the computation docking study lent support that the binding cleft for the BH3 domain of the Bak peptide on the surface of Bcl-xL is the target area for the synthetic inhibitors. Over 90% of the conformational search results showed the terephthalamide docked to this region. The overlay of the top-ranked docking result with the BH3 domain of the Bak peptide in the Bcl-xL/Bak complex suggested that the side chains of the terephthalamide scaffold have an analogous spatial arrangement to the three key alkyl side chains of the Bak peptide.

Conformational studies. The intramolecular hydrogen bond between the amide —NH and the alkoxy oxygen atom of 3-9 ensures that the 2-isopropoxy group and the upper isobutyl side chain are positioned on the same side of the terephthalamide. This hydrogen bond was confirmed by proton NMR experiments, which showed very little change in the amide —NH resonance (δ=0.54 ppm) on heating (Δδ=1.54 ppb/K) or changing concentration. As a comparison, 2-isopropylamino-terephthalamide 3-17 showed both concentration (7.36 ppm, 0.5M in CDCl₃; 6.58 ppm, 0.05M in CDCl₃; 6.46 ppm, 0.005M in CDCl₃, 298K) and temperature (A6=5.5 ppb/K) dependence of the aniline proton, suggesting inter- rather than intramolecular hydrogen bonding.

The conformation of the lower tertiary amide in 3-22 in solution was probed by computational simulations and ¹H-NMR spectroscopy. MM2 energy minimization using A macromodel suggested that the Z- conformation is favored by 8.01 kJ/mole in water solution and indeed NMR integration indicated that 72% of 3-22 adopted the Z-conformation in chloroform solution. Computer simulation showed that H_{b} and H_{c} have similar distances to the ortho-proton Hₐ in both the Z- and E-conformations (2.52 Å and 2.62 Å, respectively).

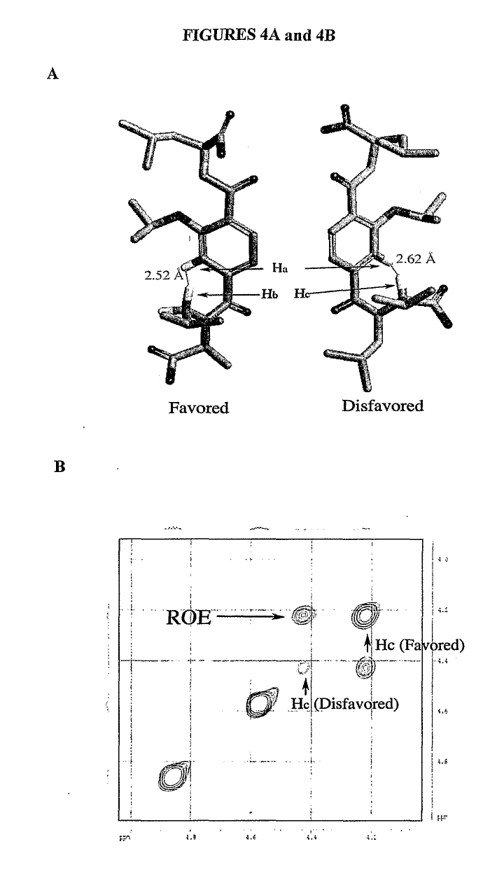

However, only the nuclear Overhauser effect (NOE) cross peaks between H_{b} and the ortho-aryl proton Hₐ were detected, while no significant NOE effect could be seen between H_{c} and Hₐ, suggesting the Z- is the major conformation in solution. Rotational Overhauser effect spectroscopy (ROESY) confirmed the presence of both Z- and E-conformations (FIG. 4A). Correlations corresponding to the chemical exchange of H_{b} and H_{c} were observed in the ROESY experiment (FIG. 4B), which indicated that both conformations of 3-22 exist in DMSO solution at 298K. Furthermore, the signals of both Hₐ and H_{b}, which are split at room temperature, coalesced at 353K. These combined experimental results suggest that both Z- and E-amide conformations are present with the Z-conformation being favored.

Disruption of Bcl-x_{L}/Bax association in whole cells. HEK293 cells were plated at an appropriate density 24 hours prior to transfection and incubated overnight. Mirus LT-1 transfection reagent (6 μl) was added dropwise into 100 μl of serum free RPMI medium and incubated at room temperature for 20 minutes. 2 μg HA-Bcl-x_{L} and 2 μg Flag-Bax pcDNA3 were added to the diluted transfection reagent and mixed by gentle pipetting. The transfection reagent/DNA complex was added dropwise to the cells and the cells were gently rocked. The cells were then incubated for 24 hours with media solution containing various concentrations of terephthalamide compounds. The cells were scraped in PBS and lysed in NP-40 lysis buffer. HA-tagged Bcl-x_{L} protein was collected via immunoprecipitation with HA antibody, washed and resuspended in Laemmli buffer (2×). The resulting mixture was loaded on to a 12.5% SDS-PAGE gel for protein separation, then transferred to nitrocellulose for western blots analysis. The presence of Bax was probed with anti-flag antibody. The inhibitory potency of the terephthalamide compounds was determined by measuring the relative intensity of Bax protein bound to Bcl-x_{L}.

### REFERENCES

(1) J. M. Adams, S. Cory, Science 1998, 281, 1322-1326; J. C. Reed, Nature 1997, 387, 773-776.
(2) D. P. Lane, Nature 1992, 358, 15-16.
(3) T. G. Graeber, C. Osmanian, T. Jacks, D. E. Housman, C. J. Koch, S. W. Lowe, A. J. Giaccia, Nature 1996, 379, 88-91; E. R. Fearon, B. Vogelstein, Cell 1990, 61, 759-767.
(4) A. Strasser, D.C. S. Huang, D. L. Vaux, Biochim. Biophys. Acta-Rev. Cancer 1997, 1333, F151-F178.
(5) D. T. Chao, S. J. Korsmeyer, Annu. Rev. Immunol. 1998, 16, 395-419.
(6) S. P. Tzung, K. M. Kim, G. Basanez, C. D. Giedt, J. Simon, J. Zimmerberg, K. Y. J. Zhang, D. M. Hockenbery, Nat. Cell Biol. 2001, 3, 183-191; I. J. Enyedy, Y. Ling, K. Nacro, Y. Tomita, X. H. Wu, Y. Y. Cao, R. B. Guo, B. H. Li, X. F. Zhu, Y. Huang, Y. Q. Long, P. P. Roller, D. J. Yang, S. M. Wang, J. Med. Chem. 2001, 44, 4313-4324; J. L. Wang, D. X. Liu, Z. J. Zhang, S. M. Shan, X. B. Han, S. M. Srinivasula, C. M. Croce, E. S. Alnemri, Z. W. Huang, Proc. Natl. Acad. Sci. U.S.A. 2000, 97, 7124-7129; A. A. Lugovskoy, A. I. Degterev, A. F. Fahmy, P. Zhou, J. D. Gross, J. Y. Yuan, G. Wagner, J. Am. Chem. Soc. 2002, 124, 1234-1240.
(7) A. Degterev, A. Lugovskoy, M. Cardone, B. Mulley, G. Wagner, T. Mitchison, J. Y. Yuan, Nat. Cell Biol. 2001, 3, 173-182.
(8) J. M. Adams, S. Cory, Trends Biochem. Sci. 2001, 26, 61-66.
(9) W. E. Stites, Chem. Rev. 1997, 97, 1233-1250.
(10) B. P. Orner, J. T. Ernst, A. D. Hamilton, J. Am. Chem. Soc. 2001, 123, 5382-5383.
(11) 0. Kutzki, H. S. Park, J. T. Ernst, B. P. Orner, H. Yin, A. D. Hamilton, J. Am. Chem. Soc. 2002, 124, 11838-11839.
(12) C. A. Lipinski, F. Lombardo, B. W. Dominy, P. J. Feeney, Adv. Drug Deliver. Rev. 1997, 23, 3-25.
(13) H. Yin, A. D. Hamilton, Bioorg. Med. Chem. Lett. 2004, 14, 1375-1379.
(14) M. Sattler, H. Liang, D. Neftesheim, R. P. Meadows, J. E. Harlan, M. Eberstadt, H. S. Yoon, S. B. Shuker, B. S. Chang, A. J. Minn, C. B. Thompson, S. W. Fesik, Science 1997, 275, 983-986.
(15) Energy minimization was carried out using MM2 forcefield within Macromodel V7.0.
(16) W. L. Jorgensen, E. M. Duffy, Adv. Drug Deliver. Rev. 2002, 54, 355-366; W. L. Jorgensen, E. M. Duffy, Bioorg. Med. Chem. Lett. 2000, 10, 1155-1158.
(17) W. L. Jorgensen, Science 2004, 303, 1813-1818.
(18) The logP and logS calculations were carried out using QikProp V2.1 (Shrodinger Inc., New York, 2003). Huuskonen et al. have shown that 85% of drugs have logS values between −1 and −5, and virtually none has a value below −6 (J. Chem. Inf. Comput. Sci. 1998, 38, 450-456).
(19) J. T. Ernst, J. Becerril, H. S. Park, H. Yin, A. D. Hamilton, Angew. Chem.-Int. Edit. 2003, 42, 535-550.
(20) Y. Cheng, W. H. Prusoff, Biochem. Pharmacol. 1973, 22, 3099-3108.
(21) P. Artursson, K. Palm, K. Luthman, Adv. Drug Deliver. Rev. 2001, 46, 27-43; J. D. Irvine, L. Takahashi, K. Lockhart, J. Cheong, J. W. Tolan, H. E. Selick, J. R. Grove, J. Pharm. Sci. 1999, 88, 28-33.
(22) G. M. Morris, D. S. Goodsell, R. Huey, A. J. Olson, J. Comput. Aid. Mol. Des. 1996, 10, 293-304; G. M. Morris, D. S. Goodsell, R. S. Halliday, R. Huey, W. E. Hart, R. K. Belew, A. J. Olson, J. Comput. Chem. 1998, 19, 1639-1662; D. S. Goodsell, A. J. Olson, Proteins: Struct. Funct. Genet. 1990, 8, 195-202

in the claims:

1. A compound or its pharmaceutically acceptable salt of the formula: 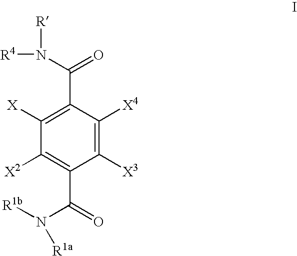
where X is H, halogen (F, Cl, Br, I), R, OR, SR or NR^{c}R^{d};
X², X³ and X⁴ are each independently selected from H, halogen, OH, R^{e} or OR^{e},
R⁴ is H, an unsubstituted or substituted C₁-C₈ alkyl or alkene, or an unsubstituted or substituted C₁-C₆ alkylene amine;
R′ is H, an unsubstituted or substituted C₁-C₈ alkyl or alkene, an unsubstituted or substituted C₁-C₆ alkylene amine, or a 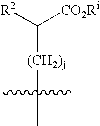
group,
where Rⁱ is H or C₁-C₄ alkyl; j is 0, 1 or 2;
R² is independently H, an unsubstituted or substituted hydrocarbon, an unsubstituted or substituted alkoxy, unsubstitued or substituted ester, an unsubstituted or substituted alkanol, an unsubstituted or substituted alkanoic acid, an unsubstituted or substituted thioester, an unsubstituted or substituted thioether, an unsubstituted or substituted amine, an unsubstituted or substituted mono- or dialkylamide, an substituted or unsubstituted alkylene amide, an unsubstituted or substituted alkyleneamine; or
R′ together with the nitrogen atom to which R′ is attached form an amino acid residue;
R is H, an unsubstituted or substituted C₁-C₁₀ alkyl or acyl group, an unsubstituted or substituted aryl, heteroaryl, alkylene aryl or alkylene heteroaryl group;
R^{C} and R^{d} are independently H, C₁-C₆ alkyl, a C₁-C₆ alkanol or a C₁-C₆ acyl group with the proviso that if one of Rc or Rd is an acyl group, the other of R^{c} or R^{d} cannot also be an acyl group;
R^{e} is an unsubstituted or substituted C₁-C₆ alkyl or acyl group, or an unsubstituted or substituted aryl or alkylene aryl group;
R^{1a} and R^{1b} are each independently H, unsubstituted or substituted C₁-C₈ alkyl or alkene, an unsubstituted or substituted aryl or alkylene aryl group, a C₁-C₆ alkylene amine which is optionally substituted with one or two C₁-C₄ alkyl groups, a 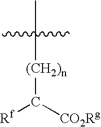
group;
Where R^{g} is H or C₁-C₆ alkyl;
n is 0, 1 or 2; and
R^{f} is H, an unsubstituted or substituted hydrocarbon, an unsubstituted or substituted alkoxy, an unsubstitued or substituted ester, an unsubstituted or substituted alkanol, an unsubstituted or substituted alkanoic acid, an unsubstituted or substituted thioester, an unsubstituted or substituted thioether, an unsubstituted or substituted amine, an unsubstituted or substituted mono- or dialkylamide, an substituted or unsubstituted alkylene amide, an unsubstituted or unsubstituted alkyleneamine or an alkyleneguanidine group; or
R^{1a} and R^{1b}, together with the nitrogen atom to which R^{1a} and R^{1b} are attached, form an amino acid residue.

2. The compound according to claim 1 wherein R⁴ is H and R′ together with the nitrogen to which R′ is attached form an α-amino acid residue.

3. The compound according to claim 1 wherein R⁴ is H and R′ together with the nitrogen to which R′ is attached form an amino acid residue obtained from alanine, arginine, asparagine, aspartic acid, cysteine, glutamine, glutamic acid, glycine, histidine, isoleucine, leucine, lysine, methionine, phenylalanine, praline, serine, threonine, tryptophan, tyrosine or valine.

4. The compound according to claim 1 wherein R² is an unsubstituted or substituted alkyl or aryl group, an unsubstituted or substituted alkoxy or ester group, an unsubstituted or substituted alkanol or alkanoic acid, an unsubstituted or substituted C₁-C₆ thioether, an unsubstituted or substituted amine, an unsubstituted or substituted alkylamide or alkylene amide or an alkyleneguanidine group.

5. The compound according to claim 1 wherein R^{1a} and R^{1b} together with the nitrogen to which they are attached form an α-amino acid residue.

6. The compound according to claim 1 wherein R^{1a} and R^{1b} together with the nitrogen to which they are attached form an amino acid residue obtained from alanine, arginine, asparagine, aspartic acid, cysteine, glutamine, glutamic acid, glycine, histidine, isoleucine, leucine, lysine, methionine, phenylalanine, praline, serine, threonine, tryptophan, tyrosine or valine.

7. The compound according to claim 1 wherein R⁴ and X form a hydrogen bond.

8. The compound according to claim 1 wherein X is H, OR, SR or NR^{c}R^{d}.

9. The compound according to claim 1 wherein X is OR.

10. The compound according to claim 8 where R is an alkyl or aryl group.

11. The compound according to claim 10 wherein R is a C₁-C₄ alkyl group.

12. The compound according to claim 1 wherein Rc and Rd are independently H, C₁-C₃ alkyl or C₁-C₃ alkanol.

13. The compound according to claim 1 wherein X², X³ and X⁴ are each independently H.

14. The compound according to claim 1 wherein X is an OR group and R is a C₁-C₃ alkyl group or an unsubstituted or substituted aryl or alkylene aryl group.

15. The compound according to claim 1 wherein R^{1a} and R^{1b} are each independently H, C₁-C₄ alkyl or an unsubstituted or substituted aryl group.

16. The compound according to claim 15 wherein said aryl group is a benzyl or unsubstituted phenyl group.

17. The compound according to claim 1 wherein R^{e} is a C₁-C₃ alkyl group.

18. The compound according to claim 1 where R′ is a 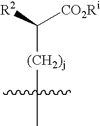
a group
wherein j is 0; Rⁱ is H or C₁-C₃ alkyl; and
R² is an unsubstituted or substituted alkyl or aryl group, an unsubstituted or substituted alkoxy or ester group, an unsubstituted or substituted alkanol or alkanoic acid, an unsubstituted or substituted C₁-C₆ thioether, an unsubstituted or substituted amine, an unsubstituted or substituted alkylamide or alkylene amide or an alkyleneguanidine group.

19. The compound according to claim 1 wherein either of R^{1a} or R^{1b}, but not both, is a 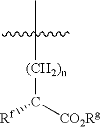
group,
wherein R^{g} H or C₁-C₄ alkyl;
n is 0, 1 or 2; and
R^{f} is H, an unsubstituted or substituted hydrocarbon, unsubstituted or substituted alkoxy, unsubstitued or substituted ester, an unsubstituted or substituted alkanol, an unsubstituted or substituted alkanoic acid, an unsubstituted or substituted thioester, an unsubstituted or substituted thioether, an unsubstituted or substituted amine, an unsubstituted or substituted alkylamide, an substituted or unsubstituted alkylene amide, an unsubstituted or unsubstituted alkyleneamine, an unsubstituted or substituted alkyleneguanidine.

20. The compound according to claim 1 wherein n is 0.

21. The compound according to claim 1 wherein R^{g} is H.

22. The compound according to claim 1 wherein R⁴ is H and X is a hydrogen bond acceptor group.

22. A pharmaceutical composition comprising an effective amount of a compound according to claim 1 in combination with a pharmaceutically acceptable carrier, additive or excipient.

23. A method of treating, preventing or reducing the likelihood of a condition or disease state in a patient, said condition or disease state being modulated through the interaction of an α-helical protein with a binding site of said protein, said method comprising administering to a patient in need of therapy an effective amount of one or more compounds according to claim 1, optionally in a pharmaceutically acceptable carrier, additive or excipient.

24. The method according to claim 23 wherein said condition or disease state is selected from the group consisting of viral infections, (including Hepatitis B virus (HBV) infections, human immunodeficiency virus (HIV) infections or conditions associated with such infections (AIDS), Herpes Simplex virus infections (HSV) infections, tumors and/or cancer, proliferative diseases including psoriasis, genital warts and hyperproliferative keratinocyte disease including hyperkeratosis, ichthyosis, keratoderma, lichen planus, hypertension, neuronal disorders, asthma, autoimmune diseases including lupus (lupus erythematosus), multiple sclerosis, arthritis, including rheumatoid arthritis, rheumatic diseases, fibromyalgia, Sjögren's disease and Grave's disease and neurodegenerative diseases including Alzheimer's disease and Parkinson's disease.

25. The method according to claim 24 wherein said viral infection is a Hepatitis B virus (HBV) infection, a human immunodeficiency virus (HIV) or a Herpes Simplex virus (HSV) infection.

26. The method according to claim 24 wherein said hyperproliferative keratinocyte disease is selected from the group consisting of hyperkeratosis, ichthyosis, keratoderma and lichen planus.

27. The method according to claim 24 wherein said autoimmune disease is selected from the group consisting of lupus erythematosus, multiple sclerosis, arthritis, rheumatic diseases, fibromyalgia, Sjögren's disease and Grave's disease.

28. The method according to claim 24 wherein said neurodegenerative disease is selected from the group consisting of Alzheimer's disease and Parkinson's disease.

29. The method according to claim 24 wherein said disease or condition is selected from the group consisting of attention deficit disorder, memory loss, language disorder and learning disorder.

30. A method of inhibiting a calmodulin dependent phosphodiesterase enzyme in a patient, said method comprising administering said patient and effective amount of a compound according to claim 1, optionally in combination with a pharmaceutically acceptable additive, carrier or excipient.

31. A method of inhibiting Bcl-X_{L} in a patient, said method comprising administering to said patient an effective amount of a compound according to claim 1 to said patient.

32. A method of inhibiting cellular invasion of a virus in a patient, said method comprising administering to said patient an effective amount of a compound according to claim 1 to said patient.

33. The method according to claim 32 wherein said virus is selected from the group consisting of HIV, HSV and HBV.
